(19) United States
(12) Patent Application Publication
(10) Pub. No.: US 2016/0310552 A1
(43) Pub. Date: Oct. 27, 2016

(54) NORTHERN WHITE KIDNEY BEAN EXTRACT AND CERATONIA SILIQUA EXTRACT IN COMBINATION WITH GREEN TEA EXTRACT IN THE TREATMENT OF EXCESS WEIGHT AND OBESITY

(71) Applicant: Grethe Stoa BIRKETVEDT, Berwin, PA (US)

(72) Inventor: Grethe Stoa BIRKETVEDT, Berwin, PA (US)

(21) Appl. No.: 15/137,967

(22) Filed: Apr. 25, 2016

Related U.S. Application Data
(60) Provisional application No. 62/152,567, filed on Apr. 24, 2015.

(51) Int. Cl. A61K 36/48; A61K 47/02; A61K 31/05; A61K 9/00; A61K 38/16; A61K 36/82; A61K 9/50

(52) U.S. Cl. CPC A61K 36/48; A61K 36/82; A61K 47/02; A61K 9/5063; A61K 9/0053; A61K 38/168; A61K 31/05

(57) ABSTRACT

This invention relates to compositions and methods for the treatment excessive weight and obesity, associated metabolic and physical aberrations such as, but not limited to an altered plasma lipid profile and elevated blood pressure. In yet other embodiments, this invention relates to compositions and methods for the treatment and prevention of irritable bowel syndrome (MS).

### CROSS-REFERENCE TO RELATED APPLICATIONS

This application claims the benefit of U.S. Provisional Application No. 62/152,567 entitled “Northern White Kidney Bean Extract And Ceratonia Siliqua Extract In Combination With Green Tea Extract In The Treatment Of Overweight And Obesity” filed Apr. 24, 2015, the contents of which are hereby incorporated herein by reference in its entirety.

### FIELD OF THE INVENTION

This invention relates to the field of treating excessive weight and obesity, associated metabolic and physical aberrations such as, but not limited to an altered plasma lipid profile and elevated blood pressure. In yet other embodiments, this invention relates to the treatment and prevention of irritable bowel syndrome (IBS).

### SUMMARY

Embodiments of the present invention are directed to compositions comprising a white kidney bean extract; a Ceratonia siliqua extract, and a green tea extract. In some embodiments, the white Kidney bean extract is Phaseolus vulgaris. In some embodiments, the Ceratonia siliqua extract is locust bean gum. In some embodiments, the green tea extract is Camellia sinensis. In some embodiments, the white kidney bean extract, Ceratonia siliqua extract, the green tea extract, or any combination thereof is water soluble. In some embodiments, green tea extract is decaffeinated.

In some embodiments, the composition further comprises a pharmaceutically acceptable carrier. In some embodiments, the pharmaceutically acceptable carrier is calcium sulfate. In some embodiments, the white kidney bean extract; Ceratonia siliqua extract; and green tea extract is encapsulated. In some embodiments, the white kidney bean extract; locust bean gum extract; and green tea extract is encapsulated in vegetable capsule. In some embodiments, the vegetable capsule has a disintegration time that is less than or equal to about 45 minutes when administered orally. In some embodiments, the composition is formulated in an immediate release form. In some embodiments, the composition is formulation in a slow release form.

In some embodiments, the composition further comprises a blueberry extract. In some embodiments, the white kidney bean extract is enriched for phaseolamin. In some embodiments, the locust bean gum is seed coated. In some embodiments, the green tea is enriched for catechol. In some embodiments, the green tea extract comprises about 20% to about 30% catechol by weight. In some embodiments, the green tea extract contains about 25% catechol by weight. In some embodiments, the green tea extract is enriched for polyphenols. In some embodiments, the green tea extract contains about 10% to about 20% polyphenols by weight. In some embodiments, the green tea extract contains about 10% to about 17% polyphenols by weight. In some embodiments, the green tea extract contains about 17% polyphenols by weight. In some embodiments, the green tea extract contains about 5% to about 10% caffeine by weight. In some embodiments the composition comprises about 100 milligrams and about 1,000 milligrams the white kidney bean extract.

In some embodiments, the composition further comprises about 100 milligrams to about 1,000 milligrams phaseolamin. In some embodiments, the composition comprises about 200 milligrams of phaseolamin.

In some embodiments, the composition comprises about 200 mg of white kidney bean extract. In some embodiments, the composition comprises about 25 milligrams to about and 250 milligrams of Ceratonia siliqua extract. In some embodiments, the composition comprises about 50 milligrams of Ceratonia siliqua extract. In some embodiments, the composition comprises about 10 milligrams to about 500 milligrams of green tea extract. In some embodiments, the composition comprises about 100 milligrams of green tea extract.

Some embodiments are directed to methods for promoting weight loss, comprising the step of administering a therapeutically effective amount of the compositions described herein to a subject in need thereof. In some embodiments, the composition is administered orally. In some embodiments, said subject has a body mass index (BMI) between about 25 kg/m² and 30 kg/m². In some embodiments, said subject has a BMI greater than 30 kg/m².

Some embodiments are directed to methods for reducing elevated blood pressure comprising the step of administrating a therapeutically effective amount of the compositions described herein to a subject in need thereof. In some embodiments, the composition is administered orally. In some embodiments, administration of said composition reduces systolic and diastolic blood pressure by at least about 5%.

Some embodiments are directed to methods for altering a plasma lipid profile comprising the step administrating a therapeutically effective amount of the compositions described herein to a subject in need thereof. In some embodiments, administration of said composition reduces plasma low-density lipoprotein (LDL), increases plasma high-density lipoprotein (HDL), increases the ratio HDL to LDL, reduces total plasma cholesterol or any combination thereof. In some embodiments, said subject has elevated plasma low-density lipoprotein (LDL), depressed plasma high-density lipoprotein HDL, a depressed HDL to LDL ratio or any combination thereof.

Some embodiments, are directed to a method of promoting gut health, comprising the step of administering a therapeutically effective amount of the compositions described herein to a subject in need thereof. In some embodiments, the composition is administered orally. In some embodiments, the subject is diagnosed with irritable bowel syndrome.

Some embodiments, are directed to a method of preventing, treating, and/or ameliorating the symptoms of irritable bowel syndrome, comprising the step of administering a therapeutically effective amount of the compositions described herein to a subject in need thereof. In some embodiments, the composition is administered orally.

Some embodiments are directed to methods of making the compositions disclosed herein, comprising combining the white kidney bean extract, Ceratonia siliqua extract, and green tea extract, wherein the white kidney bean extract, locust bean gum extract, and green tea extract, or any combination thereof, is water soluble.

### DETAILED DESCRIPTION

As used herein, “a therapeutically effective amount” is an amount sufficient to produce a therapeutic response. An effective amount may be determined with dose escalation studies in open-labeled clinical trials or bin studies with blinded trials.

As used herein, the term “pharmaceutically acceptable carrier” means a chemical composition, compound, or solvent with which an active ingredient may be combined and which, following the combination, can be used to administer the active ingredient to a subject.

A “pharmaceutically acceptable carrier”, as used herein, includes, but is not limited to, one or more of the following: excipients; surface active agents; dispersing agents; inert diluents; granulating and disintegrating agents; binding agents; lubricating agents; preservatives; physiologically degradable compositions such as gelatin and vegetable paste; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

Excessive weight and obesity are global health problems in modern society and are associated with a number of chronic health conditions including, but not limited to osteoarthritis, obstructive sleep apnea, fatty liver disease, type 2 diabetes, gallstones, reproductive and gastrointestinal cancers, hypertension, dyslipidemia, heart failure, coronary heart disease and stroke. These related diseases potentially could have been avoided by preventing excessive weight gain in overweight and obese persons with effective treatment methods.

This invention relates to the field of treating obesity, associated metabolic and physical aberrations such as altered plasma lipid profile and elevated blood pressure. Obesity is rapidly becoming a major health problem in modern society. Excessive weight and obesity are increasing in prevalence in all developing countries, and in the United States it has reached epidemic proportions. More than two thirds of the American population are either overweight or obese. Moreover, almost one third of children and adolescents in the United States are overweight or obese. 45% of overweight Americans and 67% of those who are obese are trying to lose weight using over the counter and dietary supplement weight loss products. Of the 70 million overweight and obese Americans, nearly 20 million also have hyperlipidemia. Almost one million Americans die annually from cardiovascular disease and the annual-treatment costs for cardiovascular diseases are an estimated $78.6 million. The estimated annual cost of obesity in the United States is an estimated $150 billion. The medical costs for people who are obese are, on average, about $1,429 higher than those of normal body weight.

An individual is considered overweight when his or her body mass index, (BMI) (defined as weight in kilograms divided by the square of height in meters) is greater than 25 kg/m². An individual is considered obese when his or her BMI is greater than 30 kg/m². Compared to individuals of normal weight (BMI between about 20 and 25 kg/m²), overweight (BMI between about 25 and 30 kg/m²) and obese individuals (greater than 30 kg/m²) have an increased risk of developing Type 2 diabetes, cardiovascular disease, hyperlipidemia, arthroses, cancer and other chronic diseases associated with excess fat mass in the body.

Poor nutrition is linked to hyperlipidemia, obesity, hypertension and diabetes, which contribute to the development of cardiovascular disease. Hypercholesterolemia is one of the most important diet-related risk factors for coronary heart disease. More than half of the middle-aged men and women in the United States have serum cholesterol values exceeding 200 mg/dl, values that significantly increase their risk for coronary heart disease.

Health experts agree that making lifestyle changes, including following a healthy eating pattern, reducing caloric intake, and engaging in physical activity, is the basis for achieving long-term weight loss. Because weight-loss and weight-management regimens have frequently been ineffective, effective medical interventions to manage weight gain and slow or prevent progression to obesity are needed. Obesity prevention strategies that begin in early childhood are most effective. Food education programs that teach the distinction between healthy food rich in fiber and unhealthy processed food with little, or no fiber content are also a necessary component of obesity prevention strategies.

Control of diet and exercise are cornerstones of the management of excess weight and obesity. A number of nutritional approaches and diets with different proportions of lipids, proteins and carbohydrates have been prescribed for weight loss. Initial guidance on weight loss was earlier years a restriction in saturated fats that unfortunately did not necessarily result in weight loss. Recently, a shift towards a reduction in refined carbohydrates has been a new approach to weight loss.

Several studies have indicated that fiber-rich foods and fiber supplements have moderate weight reducing effects, and may also improve the lipid profile in overweight and obese individuals. Fiber-rich foods and fiber supplements are also important in controlling or preventing hyperlipidemia. Untreated hyperlipidemia prematurely ages the body's arteries and can lead to stroke, heart attack and kidney failure. Identifying which fiber most effectively controls or prevents hyperlipidemia has been the goal of several studies.

Diets high in fiber content have frequently been used to obtain stable energy intake and avoid metabolic disorders caused by obesity. These diets also have many other health benefits, such as preventing constipation, hemorrhoids and diverticular disease as well as protecting against colon cancer. Population studies have shown that societies eating a high fiber diet have few obese individuals, while those eating a high fat, low fiber diet have many morbidly obese individuals (greater than or equal to 45 kg/m²).

A study of 203 healthy men showed that men with higher BMI ate more dietary fat and more simple carbohydrates than men with lower BMI. Consequently, the heaviest men ate fewer complex carbohydrates and less fiber in their diets. Several other studies have supported the proposition that weight gain is inversely associated with the intake of high fiber, whole-grain foods, but directly proportional to the intake of refined-grain foods. These studies indicate the importance of distinguishing whole-grain products from refined-grain products to aid in weight control, hyperlipidemia and cardiovascular disease.

Dieting is another common method used in weight-loss and weight-management regimens. There are numerous publicly-known diets. Several studies have shown that intensive nutrition intervention with diets rich in dietary fiber can lower serum cholesterol concentration by 20-30%, which may decrease the risk of coronary heart disease. Several studies have also suggested combining dietary fiber with a low fat cholesterol diet, as recommended by the American Heart Association. Dieting, however, is not always successful, and many people fail to lose weight or improve their blood lipid levels on diet alone.

In spite of all the diets that have been proposed over the years to improve health, many people still face the problem of decreased energy output and increased energy intake. The basic failure in finding the correct balance between energy intake and energy expenditure has resulted in increased weight gain, obesity and an increasing BMI. Replacing processed foods with foods rich in fiber and complex carbohydrates is a preferred solution but not at all easy to follow.

Life style modifications such as diet and exercise intervention are essential for both prevention and management of obesity, and pharmacotherapy may be considered if the intervention are ineffective. However, pharmaceutical approaches or anti-obesity drugs to weight control have had mixed results due to limited long-term success. As the weight is regained when the treatment is discontinued. The anti-obesity drugs approved and marketed over the years, mostly appetite suppressants, have now been withdrawn due to several serious side effects and health consequences. In the 1990's fenfluramine and dexfluramine were withdrawn from the market because of heart valve damage. In 2000, the European Medicines Agency (EMA) recommended the market withdrawal of several anti-obesity drugs, including phentermine, diethylpropion, and manzidol, due to an unfavorable risk to benefit ratio. Rimonabant was the first available CB1 receptor blocker available in 56 countries from 2006. However, the anti-obesity drug was never approved by the FDA and was withdrawn from the European market in 2009. Consequently, appetite suppressants are not a preferred choice. The appetite suppressant, sibutramine, approved by the FDA in 1997, was widely used for years, but this drug was withdrawn from the market on Oct. 8, 2010 due to serious cardiovascular events. Preliminary data indicated that 11.4% of the subjects receiving sibutramine had died, or had a heart attack, a stroke or cardiac arrest compared to 10% for subjects who were given the placebo treatment. Moreover, only a 5-10% weight loss was observed in individuals taking sibutramine during a 12 month period. Other pharmaceutical agents interfere with the body's energy-regulatory mechanisms and may have serious negative effects on the central nervous system through neuroendocrine mechanisms.

As discussed above, pharmaceutical approaches to weight control have had mixed results. These products, mostly appetite suppressants, have several serious side effects and health consequences. Another or additional weight-control or weight-reduction approach is to reduce the digestion of starch and the resultant production and absorption of simple sugars. Inhibiting the digestion of starch reduces carbohydrate absorption. The effective inhibition of starch breakdown and the resultant production of simple sugars that alter plasma lipid profiles and promote weight gain, has important implications in the field of weight loss. Phaseolamin, a glycoprotein found mainly in white and red kidney beans, is a known amylase inhibitor mainly responsible for the breakdown or digestion of starch. The digestion of starch, which is the main source of carbohydrates in the human diet, begins when food is chewed and mixed with saliva containing α-amylase that randomly hydrolyzes the α(1-4) glycosidic bonds of starch. Because α-amylase cannot cleave the terminal glucosidic bonds and branch points of starch, digestion in the mouth is incomplete. The action of digestion in the mouth accounts for only about 5% of the breakdown of carbohydrates. The average chain length, however, is generally reduced from several thousand to less than eight glucose units.

Commercially-available crude bean amylase inhibitors have failed to influence fecal caloric excretion. In addition, many of these commercially available amylase inhibitors cause side effects, such as diarrhea and abdominal discomfort. However, one long term published randomized placebo-controlled study has shown that only minor side effects occurred after intake of a supplement consisting of northern white kidney bean (150 mg) (Phaseolus vulgaris) mixed with an extract of Ceratonia siliqua (25 mg). The same supplement also showed an increased secretion of fat in feces measured in four subjects

In a long term study, the use of the white kidney bean extract mixed with extract of locust bean gum has been shown to have lipid controlling effects.

The present invention is directed to compositions comprising a white kidney bean extract (Phaseolus vulgaris), Ceratonia siliqua extract (locust bean gum, a seed-coat extract from locust bean gum), and a green tea extract (Camellia sinensis). In some embodiments, the composition further comprises a blueberry extract, mint extract, ginger, vitamin B₁₂, Vitamin B₆, folic acid or any combination thereof.

In some embodiments, the white kidney bean extract may be a northern white kidney bean extract. In some embodiments the white kidney bean extract is enriched for phaseolamin compared with a whole bean extract. In some embodiments, the white kidney bean extract is enriched for flavonoids compared with whole bean extract.

The compositions described herein may further comprise a pharmaceutically acceptable carrier. In some embodiments, the pharmaceutically acceptable carrier is calcium sulfate.

The compositions of the present invention may be useful as dietary supplements and may aid in weight reduction both in overweight and obese individuals. The compositions of the present invention may also help normal weight subjects in improving their quality of life by maintaining a normal, healthy weight.

The compositions of the present invention may also be useful for use in methods for preventing functional disorders of the gut. Some embodiments, are directed to a method of promoting gut health, comprising the step of administering a therapeutically effective amount of the compositions described herein to a subject in need thereof. In some embodiments, the composition is administered orally. In some embodiments, the subject is diagnosed with irritable bowel syndrome. Some embodiments, are directed to a method of preventing, treating, and/or ameliorating the symptoms of irritable bowel syndrome, comprising the step of administering a therapeutically effective amount of the compositions described herein to a subject in need thereof. In some embodiments, the composition is administered orally.

IBS is a common functional disorder of the gut with worldwide prevalence rates of 9-23%. The syndrome affects 1 in 5 people in the UK and the US rates generally in the area of 20%. Seventy percent of people with IBS have only minor attacks and most of these don't even see a doctor as the symptoms are rather mild. However, 25% have more moderate symptoms and in 5% severe symptoms occur. Symptoms are variable and include abdominal pain, bloating and bouts of diarrhea and/or constipation. Symptoms tend to come and go and further include nausea, belching, tiredness, backache, feeling of quickly full after eating, heartburn and bladder symptoms. Poor appetite and muscle pains can also quite often be connected with IBS. According to definition used, functional disorders are conditions where there is an absence of structural or biochemical abnormalities on common diagnostic tests, which could explain the symptoms. IBS is one of the most common disorders seen by physicians and in the United States alone there are between 2.4 and 3.5 million annual physician visits for IBS.

Pain and discomfort are normal symptoms and may occur in different areas of the abdomen. Pain usually comes and goes and often eases when you pass stool or wind. Many people describe the pain as a spasm or colic. The severity of the pain can vary from mild to severe, both from person to person, and from time to time in the same person.

The causes are not clear as IBS does not have one, distinct cause but several factors play a role in development of this condition. Most mentioned causes are diet, abnormal gut flora, food sensitives, lifestyle choices like smoking and drinking alcohol, drinking too much coffee or other caffeinated drinks, hormonal changes, heavy metal and chemical toxicity, stress, feelings such as anger, fear, depression and anxiety and changes in nerves that control bowel. Triggers may be carbonated drinks, sugar free gums (sorbitol), greasy food, caffeine, alcohol and some fruits and vegetables. Overeating, dairy products and chocolate are also very common triggers.

Normally food is passed along by regular contractions of the muscles in the wall of the gut. Stress or emotional upset may play a role in the hyperactivity of the nerves or the muscles of the gut as symptoms tend to become worse during times of stress or anxiety. Some people even report intolerance to certain food may be a natural cause.

However, IBS is mainly caused by diet and lifestyle and e.g having IBS with constipation gradually weakens the walls of the intestines, leading to a tendency to spasm, making the symptoms words. Pain and other symptoms may develop if the contractions become abnormal or overactive. Symptoms may follow a bad infection or heavy antibiotic use.

One of the main causes of IBS with constipation is abnormal gut flora. The health of the bowel ecosystem is the key to health in the whole body. Too little amount of the beneficial bacteria in the colon will increase bad bacteria and fungus proliferate thus contributing to digestive problems. The good bacteria in our bowels thrive on fat and are poisoned by simple sugars. Bad bacteria are fed primarily with flour and sugar and eliminating these food items from the diet will go a long way in healing the IBS.

The diet for IBS should include high amounts of fermented foods (which contribute to beneficial flora in the intestines), use of bitter vegetables and herbs (that stimulate the bile and therefore aid in the digestion of fats) and liberal consumption of foods rich in vitamin D such as butter, meats and fish, and getting ten minutes of direct sunshine every day (vitamin D is important for the health of colon).

IBS can change the lifestyle of a person completely. No cure is actually available, but suggestions such as exercise, managing stress levels and a healthy diet with regular small and light meals may relieve symptoms. It is also important to eat the food in slow motion, chew and swallow without haste. Missing meals and leaving long gaps between meals during the day can be devastating. Caffeinated drinks, some fresh fruit and alcohol can make the symptoms worse.

Most IBS sufferers are gluten intolerant and to avoid gluten (barley, rye, oats, wheat) are important. IBS sufferers may be advised to avoid all grains for a period of time until the gut heals and stay away from sorbitol, artificial sweetener found in sugar-free sweets, including chewing gum and in drink. People suffering from a lot of wind and bloating can try to increase intake of oats in their diet. Such as oat-based breakfast cereal or porridge. A tablespoon a day of linseeds can be recommended. Peppermint oil may help with bloating and wind as well as spasms.

Accordingly, this invention relates to the field of treating IBS. Irritable bowel syndrome, which is commonly abbreviated “IBS” is a common disorder that affects the large intestine. About one in five American adults will experience this condition. IBS is a chronic condition that causes abdominal pain, severe cramping, and sudden changes in bowel movements. While signs and symptoms of this condition can vary from person to person, common symptoms include abdominal pain and cramping, feeling bloated, excess gas, diarrhea and constipation, sometimes alternating between the two, mucus in the stool. While IBS is a long-term condition, there will probably be times when symptoms are worse, and when they might improve or disappear completely. This will usually depend on many different factors, such as current stress level, eating habits, or amount of exercise. Unlike with inflammatory bowel diseases, Crohn's disease and ulcerative colitis, the actual structure of the bowel is not abnormal.

Sometimes IBS can cause weight loss or weight gain at different times. Weight loss can occur because cramping pain can be severe enough to make you eat very little or not want to eat anything at all. Also, the subsequent diarrhea will prevent you from digesting necessary nutrients. All of this can lead to shedding pounds quickly.

Some embodiments, are directed to a method of promoting gut health, comprising the step of administering a therapeutically effective amount of the compositions described herein to a subject in need thereof. In some embodiments, the composition is administered orally. In some embodiments, the subject is diagnosed with irritable bowel syndrome. Some embodiments, are directed to a method of preventing, treating, and/or ameliorating the symptoms of irritable bowel syndrome, comprising the step of administering a therapeutically effective amount of the compositions described herein to a subject in need thereof. In some embodiments, the composition is administered orally. Embodiments of the present invention are directed to compositions for the treatment and prevention of IBS comprising extract of Northern White Kidney Bean (Phaseolus vulgaris), extract of Ceratonia Siliqua (Locust Bean Gum), extract of Green Tea (Camellia Sinensis), ginger extract and mint extract. In some embodiments, the composition described herein can be used to treat and/or prevent IBS. In some embodiments, the compositions described herein may be useful in helping subjects with IBS individuals in improving their quality of life by maintaining a healthy gut. The compositions of the present invention may be formulated as a slow release form or as an immediate release form. In some embodiments, the compositions of the present invention may be encapsulated in a vegetable capsule. In some embodiments, the vegetable capsule has a disintegration time of less than, or about 45 minutes when administered orally. In some embodiments, the vegetable capsule has a disintegration time of less than, or about 35 seconds when administered orally as an immediate release formulation. In some embodiments, the compositions of the present invention may be configured in an immediate or fast release form. In yet other embodiments, the compositions of the present invention may be configured in an extended release form.

In some embodiments, the compositions of the present invention may be used in methods for inducing weight loss. In some embodiments, weight loss may be achieved by inhibiting the absorption of dietary lipids and starch and increasing metabolic rate. In some embodiments, the compositions of the present invention may also prevent occurrence of gastrointestinal cancer and cancer of prostate. In some embodiments, the composition comprises a white kidney bean extract, a locust bean gum extract, and a green tea extract. In some embodiments, the white kidney bean extract is water soluble. In some embodiments, the Ceratonia siliqua extract is water soluble. In some embodiments, the green tea extract is water soluble.

The present invention provides a composition comprising a white kidney bean extract, a locust bean gum extract, and a green tea extract. In some embodiments, the white kidney bean extract is water soluble. In some embodiments, the Ceratonia siliqua extract is water soluble. In some embodiments, the green tea extract is water soluble.

In some embodiments, the white kidney bean is Phaseolus vulgaris. In a study published in British Journal of Nutrition Onakpoya et al presented a systematic review to evaluate the evidence for or against the efficacy of Phaseolus vulgaris. They identified relevant human randomized clinical trials (RTC) and found that a meta-analysis revealed a statistically non-significant difference in weight loss between Phaseolus vulgaris and placebo groups. A further meta-analysis revealed a statistically significant reduction in body fat favoring Phaseolus vulgaris over placebo (in doses from 500 to 3000 mg per day, in either a single dose or divided doses), but they could not draw any conclusion about the effects of Phaseolus vulgaris supplementation on weight and recommended larger and more rigorous trials in order to assess the effects of this herbal supplement alone. The extract of white kidney beans contains the α-amylase inhibitor phaseolamine that is inhibiting carbohydrates to be transformed to absorbable carbohydrates that can be utilized by the body. In this way, the energy from the diet is reduced and there will be less fat stored in the body. This fiber supplement has been extensively researched (in several studies. Unfortunately, this extract taken alone gives foetor-ex-ore and subjects are taken the product for a limited time only. Also, the weight lost from this extract alone is minimal.

In some embodiments, the Ceratonia siliqua extract is locust bean gum. Locust Bean Gum, also known as carob bean gum was already in 1969 evaluated for acceptable daily intake for man by the Joint FAO/WHO Expert Committee on Food Additives. The Carob Bean Gum was further evaluated in 1974 and in 1975. Carob bean gum (locust bean gum) is the material separated and variously refined from the endosperm of the seed of the carob tree. Ceratonia silique, a large leguminous evergreen that is widely cultivated in the Mediterranean area. The carbohydrate component of carob bean gum is considered to be a neutral galactomannan polymer consisting of a main chain of 1,4-linked D-mannose units with a side chain of D-galactose linkages to the polymannose chain.

The active ingredient Locust Bean Gum (Cerotonia Siliqua) has lipid reducing effects and will also inhibit ghrelin, the hormone that is known as the hunger hormone, making subjects more satiated at meals and making the satiation lasting a longer time. The Locust Bean Gum is also known as carob bean gum and is derived from the seeds of the carob tree. Locust bean gum has also been studied in humans as a potential weight lowering compound. Normal subjects and subjects with familial hypercholesterolemia were given between 8 and 30 grams per day of locust bean gum for 8 weeks, resulting in reduced total cholesterol and an improved HDL to LDL ratio. Participants reported increased gas, but it went away after a week or two, and no other harmful effects were reported. Locust bean gum has been extensively focused upon as food additives and has been studied in neonates and young infants and found to be a safe additive for its intended therapeutic use.

In yet another preferred embodiment, the green tea extract is Camellia sinensis. In some embodiments, the green tea extract is decaffeinated.

In some embodiments, the locust bean gum extract is a seed-coat extract. In some embodiments, the Ceratonia siliqua extract is water soluble. In a preferred embodiment, the Ceratonia siliqua extract is locust bean gum, seed-coat locust bean gum or a combination thereof. In some embodiments, the comprise Ceratonia siliqua extract is a seed-coat extract from Locust bean gum, an extract of Ceratonia siliqua enriched for flavonoids compared to a whole locust bean gum extract.

The present invention also provides a composition comprising a white kidney bean extract, a green tea extract, a Ceratonia siliqua extract, and further comprising vitamin B₁₂, blueberry extract, folic acid and combinations thereof.

The present invention also provides a composition comprising a white kidney bean extract, a green tea extract, a seed-coat extract from locust bean gum, and further comprising vitamin B₁₂, blueberry extract, folic acid, or a combination thereof.

In one embodiment, the compositions of the present invention comprise a white kidney bean extract that is enriched for phaseolamin as compared to a whole-bean extract. In another embodiment, the compositions of the present invention comprise a locust bean gum extract that is enriched for flavonoids compared to a whole-bean extract.

The compositions of the present invention may further comprise blueberry extract, mint extract, ginger, vitamin B₁₂, Vitamin B₆, folic acid or any combination thereof.

In some embodiments, the compositions of the present invention may comprise mint extract. In some embodiments, the mint extract is peppermint extract (Mentha×piperita). Individuals with IBS are often relieved of their discomfort with mint extract or mint oil. Therefore, mint extract is included in this composition. Peppermint is best known as a flavoring for candy, gum, lozenges and ice cream. In the kitchen, young peppermint leaves add zest to salads and can be included in soups and sauces. Peppermint is high in essential oils, vitamin A, beta-carotene, vitamins C and E, important B complex vitamins like folates, riboflavin and pyridoxine (B6) and vitamin K, as well as in dietary fiber. It is also an important source of potassium, calcium, iron, manganese and magnesium. Mint is one of the oldest herbs used in medicine. For centuries, traditional healers turned to peppermint due to its painkilling effects. Active ingredients in peppermint, such as menthol and methyl salicylate help active receptors in the brain that block the transmission of pain signals to the rest of the body. The medically sophisticated Egyptians cultivated it for use as digestive aid and stomach soother, as did the Greeks and Romans. Today, peppermint has been found to be beneficial for a variety of unpleasant reasons due to its anti-spasmodic effects. Moreover, many of the medical claims have been born out by research and claims. Studies indicate that the chemical compounds found in peppermint can help relax intestinal walls and sphincter smooth muscles, making it an effective tool in treating IBS. It has been found to soothe your intestinal tract and calm muscle contractions by blocking calcium channels in the digestive tract. Peppermint's calcium channel blocking action reduces pain caused by IBS. This is beneficial as the muscle contractions in people suffering from IBS occur too frequently causing pain and bloating. Peppermint has also been found to stimulate the gallbladder to secrete bile, which is used to digest fats. We know that our body digest carbohydrate first and then protein and fat as the last meaning it takes our digestive system longer to break down fat Therefore, peppermint can be a very useful digestive aid. Peppermint can be taken as enteric-coated capsules. As enteric-coated capsules it is released in the intestinal tract and not in the stomach.

In some embodiments, the compositions of the present invention comprise ginger. In some embodiments, the ginger is Zingiber officinale. The rhizome of the plant Zingiber officinale Roscoe, commonly known as ginger has been used for its medicinal properties for over 2000 years to calm the digestive tract. However, it has also been used as food additive and spice as well as phytomedicine. In the old days it was very well used in Chinese, Arabic and Indian cultures for its effect on nausea and dyspepsia. Ginger comes from the same family as turmeric and cardamom, Zingiberaceae, but it has a distinctive taste-brisk and peppery. The main healing effect of ginger is the rhizome's effectiveness as an antispasmodic for calming the digestive tract and expelling gas. This antispasmodic property is also mediated through calcium channel blockers, which stops the contractions from occurring, leading to pain and cramping relief. Today modern research confirms ginger's gut-soothing qualities and its antioxidant and anti-inflammatory effects and to relieve indigestion and cramps. Ginger, like peppermint Oil, also aids in digestion by stimulating the release of bile, gastric juice, and saliva, all of which help to break down food. 50 mg ginger extract added to extracts of white kidney bean, locust bean gum and decaffeinated green tea taken as a capsule three time a day before meals is recommended to relieve the symptoms of IBS. Ginger has been widely studied and has been reported to exhibit anti-inflammatory, antipyretic, antimicrobial, hypoglycemic, antimigraine and antihypertensive activities. More commonly, ginger has been traditionally used in disorders of the gastrointestinal tract as both an antidiarrheal and anticolic agent. Ongoing studies indicate that a potent compound called gingerol might also combat colorectal cancer. Research based on the effects of ginger on IBS is practically nonexistent, but the University of Maryland Medical Center reports that a study in which participants took a Chinese herbal formula that included ginger did find a reduction in the participants' IBS symptoms. Ginger is believed to contain serotonin antagonists that both improve gastric mobility and have an antispasmodic effect on the intestines, which may indicate ginger can offer relief from IBS by relaxing the intestines during an attack. Ginger is safe for most people, according to Medline Plus, but in high doses it may cause symptoms similar to those of IBS, such as nausea, diarrhea and cramping. Taking ginger in pills or with food may help reduce side-effects. The University of Maryland Medical Center recommends taking no more than 4 grams a day of ginger. In some embodiments, the amount of ginger present in the compositions described herein is about 150 mg for daily use in combination with mint extract, Locust bean gum extract, white kidney bean extract and decaffeinated green tea extract.

The compositions of the present invention may further comprise a pharmaceutically acceptable carrier. In some embodiments, one embodiment the pharmaceutically acceptable carrier is calcium sulfate.

In one embodiment, the compositions of the present invention are formulated as a vegetable capsule. In a preferred embodiment, the vegetable capsule has a disintegration time of not greater than 45 minutes when administered orally.

In one embodiment, the composition of the present invention contains at least 100 milligrams of white kidney bean extract. In one embodiment, the composition of the present invention contains between about 100 and about 1,000 milligrams white kidney bean extract, between about 150 and about 1000 milligrams of white kidney bean extract, between about 200 and about 1000 milligrams of white kidney bean extract, between about 250 and about 1000 milligrams of white kidney bean extract, between about 300 milligrams and about 1000 milligrams of white kidney bean extract, between about 350 milligrams and about 1000 milligrams of white kidney bean extract, between about 400 and about 1000 milligrams of white kidney bean extract, between about 450 milligrams and about 1000 milligrams of white kidney bean extract, between about 500 milligrams and about 1000 milligrams of white kidney bean extract, between about 550 and about 1000 milligrams of white kidney bean extract, between about 600 milligrams and about 1000 milligrams of white kidney bean extract, between about 650 milligrams and about 1000 milligrams of white kidney bean extract, between about 700 and about 1000 milligrams of white kidney bean extract, between about 750 milligrams and about 1000 milligrams of white kidney bean extract, between about 800 milligrams and about 1000 milligrams of white kidney bean extract, between about 850 and about 1000 milligrams of white kidney bean extract, between about 900 milligrams and about 1000 milligrams of white kidney bean extract, between about 950 milligrams or about 1000 milligrams of white kidney bean extract.

In one embodiment, the composition of the present invention contains about 125 milligrams of white kidney bean extract, about 175 milligrams of white kidney bean extract, about 225 milligrams of white kidney bean extract, about 275 milligrams of white kidney bean extract, about 325 milligrams of white kidney bean extract, about 375 milligrams of white kidney bean extract, about 425 milligrams of white kidney bean extract, about 475 milligrams of white kidney bean extract, about 525 milligrams of white kidney bean extract, about 575 milligrams of white kidney bean extract, about 625 milligrams of white kidney bean extract, about 675 milligrams of white kidney bean extract, about 725 milligrams of white kidney bean extract, about 775 milligrams of white kidney bean extract, about 825 milligrams of white kidney bean extract, about 875 milligrams of white kidney bean extract, about 925 milligrams of white kidney bean extract, or about 975 milligrams of white kidney bean extract. In the most preferred embodiment, the composition of the present invention contains about 200 milligrams of white kidney bean extract.

In yet another embodiment, the compositions of the present invention contain an amount of a white kidney bean extract sufficient to reduce daily carbohydrate absorption, compared to carbohydrate absorption observed in the absence of phaseolamin inhibition of α-amylase, when the composition is administered one, two, three or four times daily.

In one embodiment, the composition of the present invention contains at least 100 milligrams of phaseolamin. In one embodiment, the composition of the present invention contains between about 100 milligrams and about 1000 milligrams phaseolamin, between about 150 milligrams and about 1000 milligrams phaseolamin, between about 200 milligrams and about 1000 milligrams phaseolamin, between about 250 milligrams and about 1000 milligrams phaseolamin, between about 300 milligrams and about 1000 milligrams phaseolamin, between about 350 milligrams and about 1000 milligrams phaseolamin, between about 400 milligrams and about 1000 milligrams phaseolamin, between about 450 milligrams and about 1000 milligrams phaseolamin, between about 500 milligrams and about 1000 milligrams phaseolamin, between about 550 milligrams and about 1000 milligrams phaseolamin, between about 600 milligrams and about 1 gram phaseolamin, between about 650 milligrams and about 1000 milligrams phaseolamin, between about 700 milligrams and about 1000 milligrams phaseolamin, between about 750 milligrams and about 1000 milligrams phaseolamin, between about 800 milligrams and about 1000 milligrams phaseolamin, between about 850 milligrams and about 1000 milligrams phaseolamin, between about 900 milligrams and about 1000 milligrams phaseolamin, or between about 950 milligrams and about 1000 milligrams phaseolamin.

In one embodiment, the composition of the present invention contains about 125 milligrams of phaseolamin, about 175 milligrams of phaseolamin, about 225 milligrams of phaseolamin, about 275 milligrams of phaseolamin, about 325 milligrams of phaseolamin, about 375 milligrams of phaseolamin, about 425 milligrams of phaseolamin, about 475 milligrams of phaseolamin, about 525 milligrams of phaseolamin, about 575 milligrams of phaseolamin, about 625 milligrams of phaseolamin, about 675 milligrams of phaseolamin, about 725 milligrams of phaseolamin, about 775 milligrams of phaseolamin, about 825 milligrams of phaseolamin, about 875 milligrams of phaseolamin, about 925 milligrams of phaseolamin, or about 975 milligrams of phaseolamin. In the most preferred embodiment, the composition of the present invention contains about 200 milligrams of phaseolamin.

In one embodiment, the composition of the present invention contains at least 25 milligrams of a Ceratonia siliqua extract (Locust bean gum). In one embodiment, the composition of the present invention contains between about 25 and 250 milligrams of a Ceratonia siliqua extract, between about 75 and 250 milligrams of Ceratonia siliqua extract, between about 125 and 250 milligrams of Ceratonia siliqua extract, between about 175 and 250 milligrams of Ceratonia siliqua extract, or between about 225 and 250 milligrams of Ceratonia siliqua extract.

In one embodiment, the composition of the present invention contains about 100 milligrams of Ceratonia siliqua extract, about 150 milligrams of Ceratonia siliqua extract, or about 200 milligrams of Ceratonia siliqua extract. In the most preferred embodiment, the composition of the present invention contains about 50 milligrams of Ceratonia siliqua extract.

In some embodiments, the Ceratonia siliqua extract is a seed-coat Locust bean gum. In one embodiment, the composition of the present invention contains at least 25 milligrams of a seed-coat Locust bean gum. In one embodiment, the composition of the present invention contains between about 25 and 250 milligrams of a seed-coat Locust bean gum, between about 75 and 250 milligrams of seed-coat Locust bean gum, between about 125 and 250 milligrams of seed-coat Locust bean gum, between about 175 and 250 milligrams of seed-coat Locust bean gum, or between about 225 and 250 milligrams of seed-coat Locust bean gum.

In one embodiment, the composition of the present invention contains about 100 milligrams of seed-coat Locust bean gum, about 150 milligrams of seed-coat Locust bean gum, or about 200 milligrams of seed-coat Locust bean gum. In the most preferred embodiment, the composition of the present invention contains about 50 milligrams of seed-coat Locust bean gum.

In one embodiment, the composition of the present invention contains at least 10 milligrams of green tea extract. In one embodiment, the composition of the present invention contains between about 10 milligrams and about 500 milligrams green tea extract, between about 25 and 500 milligrams green tea extract, between about 75 milligrams and about 500 milligrams green tea extract, between about 125 milligrams and about 500 milligrams green tea extract, between about 175 milligrams and about 500 milligrams green tea extract, between about 225 milligrams and about 500 milligrams green tea extract, between about 275 milligrams and about 500 milligrams green tea extract, between about 325 milligrams and about 500 milligrams green tea extract, between about 375 milligrams and about 500 milligrams green tea extract, between about 425 milligrams and about 500 milligrams green tea extract, or between about 475 milligrams and about 500 milligrams green tea extract. In some embodiments, the green tea extract is decaffeinated.

In one embodiment, the composition of the present invention contains about 50 milligrams green tea extract, about 150 milligrams green tea extract, about 200 milligrams green tea extract, about 250 milligrams green tea extract, about 300 milligrams green tea extract, about 350 milligrams green tea extract, about 400 milligrams green tea extract, or about 450 milligrams green tea extract. In the most preferred embodiment, the composition of the present invention contains about 100 milligrams green tea extract.

In some embodiments, the white kidney bean extract is enriched for phaseolamin. In some embodiments, the locust bean gum is seed coated. In some embodiments, the green tea is enriched for catechol. In some embodiments, the green tea extract comprises about 20% to about 30% catechol by weight. In some embodiments, the green tea extract contains about 25% catechol by weight. In some embodiments, the green tea extract is enriched for polyphenols. In some embodiments, the green tea extract contains about 10% to about 20% polyphenols by weight. In some embodiments, the green tea extract contains about 10% to about 17% polyphenols by weight. In some embodiments, the green tea extract contains about 17% polyphenols by weight. In some embodiments, the green tea extract contains about 5% to about 10% caffeine by weight. In some embodiments, the green tea extract contains about 1% caffeine by weight. In some embodiments the composition comprises about 100 milligrams and about 1,000 milligrams the white kidney bean extract.

In some embodiments, the white kidney bean extract is enriched for phaseolamin. In some embodiments, the locust bean gum is seed coated. In some embodiments, the mint extract is enriched with vitamins A, C and E, the B-complex vitamins, potassium or any combination thereof. In some embodiments, the green tea extract is enriched for catechols. In some embodiments, the green tea extract comprises about 20% to about 30% catechols by weight. In some embodiments, the green tea extract contains about 25% catechols by weight. In some embodiments, the green tea extract is enriched for polyphenols. In some embodiments, the green tea extract contains about 10% to about 20% polyphenols by weight. In some embodiments, the green tea extract contains about 10% to about 17% polyphenols by weight. In some embodiments, the green tea extract contains about 17% polyphenols by weight. In some embodiments, the green tea extract is decaffeinated and contains less than 1% caffeine by weight.

In some embodiments, the compositions described herein further comprise about 100 milligrams to about 1,000 milligrams phaseolamin. In some embodiments, the compositions comprise about 200 milligrams of phaseolamin.

Embodiments of the present invention are directed to weight loss compositions. As such embodiments of the present invention are directed to compositions comprising an extract from white kidney bean, an extract from Ceratonia Siliqua, and an extract from green tea. Preferably, the white kidney bean extract is water soluble. Preferably, the extract from Ceratonia Siliqua is water soluble, preferably the extract from green tea is water soluble. In a preferred embodiment the white kidney bean is Phaseolus vulgaris. In another preferred embodiment, the Ceratonia Siliqua is locust bean gum. In yet another preferred embodiment, the green tea is Camellia sinensis. The present invention also provides a composition comprising an extract from white kidney bean, an extract from Ceratonia siliqua (a seed-coat extract from Locust bean gum), an extract from green tea, vitamin B₁₂, Vitamin B₆, blueberry extract, and/or folic acid and/or combinations thereof. The present invention also provides a composition comprising an extract from white kidney bean, an extract from Ceratonia siliqua (a seed-coat extract from Locust bean gum), an extract from green tea, mint extract, ginger extract and/or combinations thereof. In some embodiments, the compositions described herein may be formulated in a powder form for immediate release upon ingestion. The present invention also provides a composition comprising an extract from white kidney bean, an extract from green tea, an extract from Ceratonia siliqua (a seed-coat extract from locust bean gum) that is in powder form as an immediate release form. The present invention also provides a composition comprising an extract from white kidney bean, an extract from green tea, an extract from Ceratonia siliqua (a seed-coat extract from locust bean gum) and further comprising vitamin B₁₂, Vitamin B₆, blueberry extract, folic acid, or any combination thereof.

Some embodiments, are directed to a method of promoting gut health, comprising the step of administering a therapeutically effective amount of the compositions described herein to a subject in need thereof. In some embodiments, the composition is administered orally. In some embodiments, the subject is diagnosed with irritable bowel syndrome. Some embodiments, are directed to a method of preventing, treating, and/or ameliorating the symptoms of irritable bowel syndrome, comprising the step of administering a therapeutically effective amount of the compositions described herein to a subject in need thereof. In some embodiments, the composition is administered orally. The present invention also provides a composition for use in treating, ameliorating the symptoms of, or preventing IBS, comprising an extract from white kidney bean, an extract from Ceratonia siliqua (a seed-coat extract from Locust bean gum), an extract from green tea, mint extract, and ginger extract. The composition is in powder form as an immediate release form.

In one embodiment, the compositions of the present invention comprise a white kidney bean extract that is enriched for phaseolamin as compared with a whole-bean extract. In another embodiment, the compositions of the present invention comprise a seed-coat extract from Locust bean gum, an extract of Ceratonia siliqua enriched for flavonoids compared to a whole locust bean gum extract. The compositions described herein may further comprise a pharmaceutically acceptable carrier. In one embodiments, the pharmaceutically acceptable carrier is calcium sulfate. In one embodiments, the compositions of the present invention may be encapsulated in a vegetable capsule. In a preferred embodiment, the vegetable capsule has a disintegration time of less than, or about 45 minutes when administered orally.

Embodiments of the present invention are directed to compositions for the treatment, amelioration of the symptoms of, or prevention of IBS. As such, embodiments are directed to a composition comprising a white kidney bean extract; a locust bean gum extract and a green tea extract combined with mint extract and ginger extract. In some embodiments, the white Kidney bean is Phaseolus vulgaris. In some embodiments, the Ceratonia siliqua is Locust bean gum. In some embodiments, the green tea is Camellia sinensis. In some embodiments, the mint extract is a peppermint extract (Mentha×piperita). In some embodiments, the ginger extract is Zingiber officinale. In some embodiments, the white kidney bean extract, locust bean gum extract, the green tea extract, or any combination thereof mentioned above is water soluble. Some embodiments further comprise a pharmaceutically acceptable carrier. In some embodiments, the pharmaceutically acceptable carrier is calcium sulfate. In some embodiments, the white kidney bean extract; locust bean gum extract; and green tea extract is encapsulated with mint extract and ginger extract. In some embodiments, the white kidney bean extract; locust bean gum extract; and green tea extract is encapsulated in vegetable capsule. In some embodiments, the vegetable capsule has a disintegration time that is less than or equal to about 45 minutes when administered orally. In some embodiments, the composition is formulated in an immediate release form. In some embodiments, the composition further comprises a pharmaceutically acceptable carrier. In some embodiments, the pharmaceutically acceptable carrier is calcium sulfate. In some embodiments, the white kidney bean extract; locust bean gum extract; the green tea extract; and the mint extract is encapsulated. In some embodiments, the white kidney bean extract; locust bean gum extract; mint extract; and green tea extract is encapsulated in vegetable capsule. In some embodiments, the vegetable capsule has a disintegration time that is less than or equal to about 45 minutes when administered orally. In some embodiments, the composition is formulated in an immediate release form.

Embodiments of the present invention are directed to compositions comprising a white kidney bean extract; a locust bean gum extract, decaffeinated green tea extract, mint extract, and ginger. In some embodiments, the white Kidney bean is Phaseolus vulgaris. In some embodiments, the Ceratonia siliqua is the Locust bean gum. In some embodiments, the green tea is decaffeinated and is Camellia sinensis. In some embodiments the mint extract is menthe spp. In some embodiments, the white kidney bean extract, the Ceratonia siliqua extract, the green tea extract, and the mint extract and ginger extract or any combination thereof is water soluble.

The present invention provides a method for promoting weight loss comprising the step of administering a therapeutically effective amount of a composition of the present invention to a subject in need thereof, wherein the administration of said composition promotes weight loss. In one embodiment, the composition of the present invention is administered orally.

In one embodiment, the compositions of the present invention are administered to an overweight subject with a BMI between about 25 kg/m² and 30 kg/m². In another embodiment, the compositions of the present invention are administered to an obese subject with a BMI greater than 30 kg/m².

The present invention provides a method for reducing elevated blood pressure comprising the step of administering a therapeutically effective amount of the composition of the present invention to a subject in need thereof, wherein the administration of said composition reduces elevated blood pressure. In one embodiment, the composition of the present invention is administered orally. In one embodiment, administration of a composition of the present invention reduces systolic and/or diastolic blood pressure by at least 6%, 7%, 8%, 9%, 10%, 11%, 12%, 13%, 14%, 15%, 16%, 17%, 18%, 19%, 20%, 21%, 22%, 23%, 24%, or 25%. In one embodiment, administration of a composition of the present invention reduces systolic and/or diastolic blood pressure between about 7 and about 25%, about 10 and about 25%, about 12 and about 25%, about 15 and about 25%, about 17 and about 25%, about 17 and about 25%, about 20 and about 25%, or about 22 and about 25%.

In one embodiment, administration of a composition of the present invention reduces systolic and/or diastolic blood pressure about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In the most preferred embodiment, administration of a composition of the present invention reduces systolic and/or diastolic blood pressure by at least 5%, between about 5 and about 25%, or about 5%.

In another embodiment, administration of a composition of the present invention reduces systolic and/or diastolic blood pressure by at least 5 mmHg. In a more preferred embodiment, administration of a composition of the present invention reduces systolic and/or diastolic blood pressure by about 5, 6, 7, 8, 9, 10, 11, 12, 13, 14, 15, 16, 17, 18, 19, 20, 21, 22, 23, 24 or 25 mmHg.

In yet another embodiment, administration of a composition of the present invention reduces systolic blood pressure to less than 140 mmHg and/or diastolic blood pressure to less than 90 mmHg. In a more preferred embodiment, administration of a composition of the present invention reduces systolic blood pressure to less than 130 mmHg and/or diastolic blood pressure to less than 80 mmHg.

In one embodiment, the present invention provides a method for altering a plasma lipid profile comprising the step of administering a therapeutically effective amount of the composition of the present invention to a subject in need thereof, wherein the administration of said composition alters the plasma lipid profile.

In one embodiment, the subject in need thereof has elevated low-density lipoprotein (LDL). In another embodiment, the subject in need thereof has depressed high-density lipoprotein (HDL). In yet another embodiment, the subject in need thereof has a depressed HDL to LDL ratio.

In a preferred embodiment, the present invention provides a method for altering a plasma lipid profile comprising the step of administering a therapeutically effective amount of the composition of the present invention to a subject in need thereof, wherein the administration of said composition reduces plasma LDL.

In another preferred embodiment, the present invention provides a method for altering a plasma lipid profile comprising the step of administering a therapeutically effective amount of the composition of the present invention to a subject in need thereof, wherein the administration of said composition alters the plasma lipid profile, wherein administration of said composition increases plasma HDL.

In another preferred embodiment, the present invention provides a method for altering a plasma lipid profile comprising the step of administering a therapeutically effective amount of the composition of the present invention to a subject in need thereof, wherein the administration of said composition alters the plasma lipid profile, wherein administration of said increases the ratio of HDL to LDL.

In another embodiment, the present invention provides a method for altering a plasma lipid profile comprising the step of administering a therapeutically effective amount of the composition of the present invention to a subject in need thereof, wherein the administration of said composition alters the plasma lipid profile, wherein administration of said composition reduces total plasma cholesterol.

In another embodiment, the present invention provides a method for preventing IBS and/or relieve symptoms of IBS, comprising the step of administering a therapeutically effective amount of the compositions described herein to a subject in need thereof. In some embodiments, the composition is administered orally.

In another embodiment, the present invention provides a method treating and/or preventing IBS comprising the step of administering a therapeutically effective amount of a composition of the present invention to a subject in need thereof. In some embodiments, the composition is administered orally.

The present invention provides a method of making the composition of the present invention, comprising the steps of providing a white kidney bean extract, a Ceratonia silique extract, and a green tea extract and combining said extracts. Preferably, the white bean extract is water soluble. Preferably, the Ceratonia siliqua extract is water soluble. Preferably, the green tea extract is water soluble. In a preferred embodiment, the white kidney bean is Phaseolus vulgaris. In another preferred embodiment the Ceratonia siliqua is Locust bean gum. In yet another preferred embodiment, the green tea is Camellia sinensis. In one embodiment, the white kidney bean extract that is enriched for phaseolamin as compared to a whole-bean extract. In another embodiment, the compositions of the present invention is enriched for flavonoids compared to a whole-bean extract.

Some embodiments are directed to a method of making the compositions of the present invention, comprising combining the white kidney bean extract, locust bean gum extract, and green tea extract, with mint extract, and ginger extract wherein the white kidney bean extract, locust bean gum extract, and green tea extract, or any combination thereof, is water soluble.

Some embodiments are directed to methods of making the compositions disclosed herein, comprising combining the white kidney bean extract, locust bean gum extract, and green tea extract, wherein the white kidney bean extract, locust bean gum extract, and green tea extract, or any combination thereof, is water soluble.

The Ceratonia silique extract provided in the production of the compositions of the present invention is preferably seed-coat extract from locust bean gum. Preferably, the seed-coat extract from a Ceratonia silique extract is water soluble.

In another embodiment of the present invention, vitamin B₁₂, vitamin B₆, blueberry extract, folic acid or any combinations thereof are further provided and combined with the white kidney bean extract, Ceratonia siliqua extract, and green tea extract.

In another embodiment of the present invention, vitamin B₁₂, vitamin B₆, blueberry extract, folic acid or any combinations thereof are further provided and combined with the white kidney bean, green tea, and a seed-coat extract of Ceratonia siliqua.

In yet another embodiment of the present invention, a pharmaceutically acceptable carrier is further provided and combined with the compositions of the present invention. In one embodiment the pharmaceutically acceptable carrier is calcium sulfate.

In another embodiment, the green tea extract provided in the production of the compositions of the present invention is enriched for catechol compared to a whole tea-leaf extract. In one embodiment, the green tea extract contains between about 2% and 100% catechol by weight, between about 5% and about 100% catechol by weight, between about 10 and about 100% catechol by weight, between about 20% and about 100% catechol by weight, between about 30 and 100% catechol by weight, between about 10% and about 100% catechol by weight, between about 50 and 100% catechol by weight, between about 5% and about 100% catechol by weight, or between about 20% and about 50% catechol by weight. In a more preferred embodiment the green tea extract contains between about 20% and about 30% catechol by weight.

In one embodiment the green tea extract contains about 2% catechol by weight, about 5% catechol by weight, about 10% catechol by weight, about 15% catechol by weight, or about 20% catechol by weight, about 30% catechol by weight. In the most preferred embodiment, the green tea extract contains about 25% catechol by weight.

In one embodiment, the green tea extract provided in the production of the compositions of the present invention is enriched for polyphenols. In one embodiment, the green tea extract contains between about 2% and about 100% polyphenols, between about 5% and about 100% polyphenols, between about 10% and about 100% polyphenols, between about 20% and about 100% polyphenols, between about 30% and about 100% polyphenols, between about 50% and about 100% polyphenols, between about 10% and about 50% polyphenols, or between about 10% and about 25% polyphenols. In a more preferred embodiment, the green tea extract contains between about 10% and about 20% polyphenols by weight.

In one embodiment, the green tea extract contains about 2% polyphenols, about 5% polyphenols, about 10% polyphenols, about 15% polyphenols, about 20% polyphenols, about 25% polyphenols, about 30% polyphenols, about 50% polyphenols, or about 75% polyphenols. In the most preferred embodiment, the green tea extract contains about 17% polyphenols by weight.

In one embodiment, the green tea extract provided in the production of the compositions of the present invention contains between about 2% and 100% caffeine by weight, between about 5 and 100% caffeine by weight, about 10% and 100% caffeine by weight, between about 20 and 100% caffeine by weight, about 30% and 100% caffeine by weight, between about 40 and 100% caffeine by weight, between about 50% and 100% caffeine by weight, about 60 and 100% caffeine by weight, between about 70% and 100% caffeine by weight, between about 80% and 100% caffeine by weight, about 90% and 100% caffeine by weight, between about 5% and 50% caffeine by weight, between about 5% and 25% caffeine by weight, or between about 20% and 30% caffeine by weight. In the most preferred embodiment, the green tea extract provided in the production of the compositions of the present invention contains between about 5% and 10% caffeine by weight. In some embodiments, the green tea extract provided in the production of the compositions of the present invention is decaffeinated. In some embodiments, the green tea extract provided in the production of the compositions of the present invention contains about 1% caffeine by weight. In some embodiments, the green tea extract provided in the production of the compositions of the present inventions is decaffeinated.

The human body expends energy metabolism, muscular work and thermogenesis. This expenditure is compensated for by the energy supplied by the assimilation of foods. If the amount of energy supplied from the dietary foods is identical to the amount of energy a person expends, the individual will maintain a stable weight. If there is an excess supply of energy, the body stores this energy in the forms of fat and gains weight. If there is a deficit in the amount of energy ingested, the body starts to draw the energy it lacks by burning off the fats stored, and the person will lose weight. Often, however, when a body is faced with an energy deficit, the body reacts to save energy and reduce thermogenesis. This is the control mechanisms which accounts for the failure of weight-reducing diets. Specifically, after losing weight for a few weeks, the person's weight stabilizes, and if he or she wishes to lose further weight, he or she will need to reduce the food intake.

Various chemical substances stimulate thermogenesis, such as nicotine, ephedrine, aspirin, caffeine etc. None of these substances have made it possible to produce a medicinal product for treating obesity since the doses required to obtain an increase in thermogenesis entail considerable side effects, which are incompatible with a treatment which is necessarily long-lasting generally extended over several months.

Reducing calorie intake may be achieved by decreasing and inhibiting the absorption of carbohydrates. Specifically, decreasing the digestion of starch reduces the production of simple sugars that are a major calorie source. Reducing starch metabolism may be achieved by inhibiting α-amylase—an enzyme responsible for the digestion of starch. Phaseolamin, a glycoprotein found mainly in white kidney bean, is an effective α-amylase inhibition. Whole, dried non-genetically modified organism (nGMO) of Northern White Kidney beans are the preferred source for phaseolamin, but other species and sources of phaseolamin may also be used. The dried beans are milled and suspended placed in aqueous solution.

Phaseolamin may be extracted from the bean by milling and suspending the milled beans in aqueous solution, followed by one or more extraction and purification cycles using methods well-known in the art, such as affinity chromatography. Extracted phaseolamin may be dried by any number of methods, including spray drying and tested for bacterial contamination, mesh (i.e., particle size), moisture content, potency, and organoleptics (i.e. physical characteristics, such as, color, taste, odor, powder, and liquid). Each of these properties may be altered and adjusted by methods well-known in the art.

In some embodiments, the Ceratonia siliqua extract is locust bean gum. Locust bean gum is also known as carob bean gum, carubin or algarroba. It is used as a thickener, stabilizer, emulsifier and gelling agent, and approved in most areas of the world for these purposes (e.g. European Union, United States of America, Japan and Australia).

The carbohydrate component of carob bean gum is considered to be a neutral galactomannan polymer consisting of a main chain of 1,4 linked D-mannose units with a side chain of D-galactose linkages to the polymannose chain. Locust bean gum has lipid reducing effects and also inhibits ghrelin, the hormone that is known as the hunger hormone, making subjects more satiated at meals and making the satiation lasting a longer time thus delaying ingestion of the next meal. The lipid lowering effects are thought to be facilitated through binding to bile acids and increasing bile acid secretion. Additionally, Locust bean gum interferes with micellar formation, which impacts cholesterol absorption. Locust bean gum fiber may also act as a water-holding and cation-exchange agent, increasing total fecal output because of the increased water-holding capacity.

Locust bean gum was evaluated as early as in 1969 for acceptable daily intake for man by the Joint FAO/WHO Expert Committee on Food Additives and further evaluated in 1974 and in 1975. It has been studied in humans as a potential lipid lowering compound. Normal subjects and subjects with familial hypercholesterolemia were given between 8 and 30 grams per day of locust bean gum for 8 weeks, resulting in reduced total cholesterol and an improved HDL to LDL ratio. Locust bean gum has been extensively focused upon as food additives and has been studied in neonates and young infants and found to be a safe additive for its intended therapeutic use.

Locust bean gum is obtained from the endosperm of the seed of the carob (locust) tree, Ceratonia siliqua (L.) Taub (Family Leguminosae). The isolated endosperm is ground to fine particle size powder, which is the locust (carob) bean gum. The gum may be further clarified as follows: The carob bean gum is dispersed in water and dissolved by heating. This solution is filtered to remove insoluble material. From this clear solution, the carob bean gum is precipitated with isopropanol or ethanol. The precipitate is then filtered, dried and ground to fine particle size powder. The specifications are set forth at the 67^{th} JECFA (2006).

Carob bean gum mainly consists of the high molecular weight (˜50,000-3,000,000) polysaccharides composed of galactomannans (I). The gum is a white to yellowish white, nearly odorless powder. The mannose:galactose ratio of locust bean gum is approximately 4:1. The structure of galactomannan is shown as structure (I)

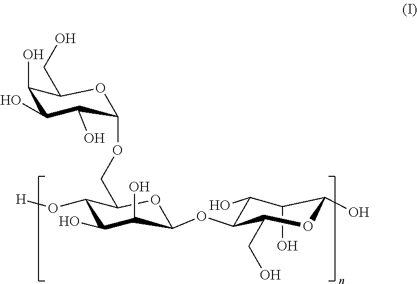

The possible non-microbiological impurities are: husk, the germ, residual amounts of ethanol or isopropanol for washing or extraction solvent (during manufacture).

In some embodiments, the Ceratonia siliqua extract may be obtained from carob kernels. In some embodiments, the carob kernels are peeled without damaging the endosperm and the germ using special processes (1): (i) Acid peeling process: The kernels are treated with sulfuric acid at a certain temperature to carbonize the seed coat. The carob gum produced with acid peeling process is whitish. (ii) Thermal peeling process: The kernels are roasted in a rotating furnace where the seed coat ‘pops off’ from the rest. The carob gum produced with thermal peeling process is somewhat darker. The isolated endosperm is ground to fine particle size powder, which is the locust (carob) bean gum. The gum may be further clarified as follows: The carob bean gum is dispersed in water and dissolved by heating. This solution is filtered to remove insoluble material. From this clear solution, the carob bean gum is precipitated with isopropanol or ethanol. The precipitate is then filtered, dried and ground to fine particle size powder.

Green tea has been used for thousands of years in Asia as both a beverage and an herbal medicine. Over the past few years, dozens of studies have been conducted on its antioxidative and chemoprotective effects. Research has shown green tea to be effective against a number of conditions, ranging from lowering cholesterol and capturing free radicals to reducing the risks of certain types of cancers. Green tea extract has been shown to decrease weight in overweight subjects. Substances which are abundant in green tea extracts may promote weight loss and treat prostatitis, a painful urinary condition.

After harvesting, the leaves of may be treated two ways. Subjecting the leaves to a fermentation process, transforming the chemical substances they contain, particularly the catechol, produces black tea. Drying the leaves immediately produces green tea.

Green tea is rich in polyphenol compounds called catechins, of which epigallocatechin-3-gallate (EGCG) is the best-studied and has shown the greatest range of beneficial effects. In addition to catechol, tea contains caffeine, the diuretic effect of which is well known. The diuretic effect is the reason for the traditional use of green tea as a medicinal plant to promote the elimination of water by the kidneys, either in the case of urinary disorders or as a supplement to weight reducing diets. The presence of caffeine is also the reason for the traditional use of tea in conditions of fatigue (asthenia). Epidemiological studies carried out on certain populations have demonstrated the beneficial effects of the chronic ingestion of tea, and more particularly of green tea.

Studies involving long-term consumption of green tea have shown anti-atherogenic effects. These effects are related to the hypocholesterolemic effects shown in several studies. Additionally, these effects are also related to ability of green tea to prevent the oxidation of LDLs in the circulation. Green tea is also known for its anti-mutagenic and anti-carcinogenic effects. It has been shown to reduce the risk of colorectal, skin cancer and breast cancer in several published studies.

As a diuretic, the use of green tea traditionally occurs in the form of infusions, liquid extracts in gel capsules or tables. In those various forms, the green tea, often combined with another diuretic plant, is generally used at a dose corresponding one to three grams of plant per day.

In the present invention, the extract of green tea contains from 20% to 30% by mass of catechol expressed as epigallocatechol gallate (EGCG). The content of catechol, expressed as EGCG is, for example, determined by methods known in the art. The extract of green tea contains from 5% to 10% by mass of caffeine.

The dose of the green tea extract chosen may be based on the average daily calorie intake, based on an analysis of food diaries over a ten-days period. Generally, one milligram green tea extract is used per nine calories. For example, 300 milligrams of a green tea extract are appropriate for a 2,700 calorie per day diet.

Blueberries are small, blue-purple fruit that belong to the genus vaccinium which also includes cranberries and bilberries. Due to the antioxidant and anthocyanin content the blueberries are effective in protecting the liver, especially reducing liver fat build-up which is in excess in overweight and obese individuals. Research have also found the content of blueberries to support cardiovascular health and reduce cognitive decline. The small blue berries may play a role in promoting the growth of nervous tissue and reduce neurological inflammation. The optimal range of isolated anthocyanin supplementation is 500-1,000 mg.

Folate has important beneficial effects on endothelial function. Previous studies have shown that women who consume ample amounts of folic acid every day had the lowest risk of hypertension. 93,803 younger women aged 27 to 44 years were studied and compared with 62.260 older women aged 43 to 70 years in the Nurses' Health Study (1990-1998. The result showed that a higher total folate intake was associated with decreased risk in incident hypertension, particularly in young women. Specifically, it is recommended that every woman should get about 700 micrograms daily to boost blood-vessel health and reduce blood pressure. Asparagus spears, artichokes and spinach, fortified pasta, breads and cereals are good folic acid sources.

Typically, obese patients are on a poor diet and have a tendency to eat foods rich in the amino acid homocysteine. Homocysteine is an amino acid in the blood and it has been found that people with moderate to high concentrations of homocysteine and/or homocystinuria, may have increased risk of thromboembolic events, especially stroke. A supplement with folate and Vitamin B 12 is important for maintaining normal function of the brain and the nervous system, and for the formation of blood. It is normally involved in the metabolism of every cell in the human body, especially affecting DNA synthesis and regulation, and also involved in fatty acid metabolism and amino acid metabolism. Vitamin B₁₂ may reduce the levels of homocysteine and thus reduce the risk of stroke. Additionally, Vitamin B₁₂ deficiency is extremely common. Hyperhomocysteinemia is caused by deficiencies in vitamins B₆, B₁₂ and folic acid. The adverse vascular and neurotoxic effects of homocysteine are associated with excess free radical generation (oxidative stress). In previous studies, higher levels of Vitamin B₁₂ have been associated with lower levels of homocysteine. Homocysteine has been linked to stroke. Importantly, folic acid and B₁₂ vitamins may lower plasma homocysteine. Studies have shown that vitamin B₁₂ has a protective effect in vascular events. Specifically, Vitamin B₁₂ may play a key role in lowering total plasma homocysteine, thus preventing subsequent vascular events in patients who have had a non-disabling stroke. Patients in this study by Spence received 2.5 mg folate, and 400 mcg of vitamin B₁₂.

A recent meta-analysis of data from 500 stroke events in prospective studies, and 1000 stroke events in retrospective trials, identified a statistically significant positive association between homocysteine levels and stroke in all age groups, independent of smoking, cholesterol and blood pressure.

Another study identified the association between elevated homocysteine levels and other risk factors for stroke and the risk of aortic atheroma progression. Fifty-seven stroke patients and twenty-one patients with transient ischemic attack underwent multiplanar transesophageal echocardiograms within one month of symptom onset and again after nine months. Aortic atheroma was graded and stratified. Use of anticoagulant, antiplatelet, and hypolipidemic drugs, and clinical and aetiological subtypes of stroke were recorded and compared in patients stratified for the presence or absence of atheroma progression. The only factors that significantly correlated with atheroma progression were homocysteine levels of 14.0 μmol/L or greater, total anterior cerebral infarct, and large-artery atherosclerosis.

It is known that silent brain infarcts and white matter lesions are associated with increased risk of both stroke and dementia. Other recently published data from the Rotterdam Scan study, a population-based study of 1,077 people aged 60-90 years, who had cerebral magnetic resonance imaging, showed that the overall risk of having either silent brain infarcts or severe white matter lesions was strongly associated with elevated homocysteine levels.

Specifically, twenty percent of the population had one or more silent brain infarcts, 80% had periventricular white matter lesions, and 92% subcortical lesions. Silent brain infarcts were 2.5 times more common in the top quintile of homocysteine concentrations (less than 13.8 μmol/L) compared with the bottom quintile (less than 8.5 μmol/L). The risk of silent brain infarct increased by 24% per standard deviation increase of homocysteine. The relationship was continuous and graded, with no obvious threshold below which homocysteine levels were not associated with risk of disease.

A recently published follow-up for 5 years of 369 healthy subjects from the Canadian Study of Health and Aging, showed that the odds ratio of developing vascular dementia/cognitive impairment or fatal stroke was 2.42 for persons with serum folate within the lowest quartile at baseline.

Estimation of dietary intake of vitamins is less accurate and sensitive than determination of plasma/serum levels. However, an association between calculated folate intake and plasma/serum levels was recently demonstrated. In a follow-up study within the Third National Health and Nutrition Examination Survey (NHANES I) dietary intake of folate was assessed at baseline among 9,764 US men and women aged 25-74 years and free of cardiovascular disease. A 24-hour dietary recall was used. Over an average of 19 years of follow-up, 926 incident stroke events and 3,758 incident cardiovascular events were documented. The occurrence rate for incident stroke for subjects with folate intake within the highest quartile at baseline was calculated to be 0.79 and 0.86 for incident cardiovascular events compared with subjects within the lowest quartile of folate intake. The calculated median-folate intake in the highest quartile was 405 μg/day, and in the lowest 99 μg/day.

Large intervention studies with homocysteine-lowering therapy, designed to show the effect of prevention of stroke occurrence in a general population (or recurrence in patients) are ongoing. An increasing number of intervention studies using surrogate endpoints, such as effects on coagulation factors, and effect on intima plaque formation, in order to assess the effect on coagulation and atherogenesis, have been published. These studies have demonstrated an effect of homocysteine-lowering therapy on both coagulation and the rate of progression of plaque formation. Elevated plasma homocysteine (hyperhomocysteinemia) is now recognized as a strong, independent risk factor for stroke and dementia.

Elevated plasma homocysteine, however, is a reversible risk factor. Consumption of foods containing B vitamins and supplementation with folic acid and vitamins B₆ and B₁₂ are the primary preventive and therapeutic treatments. The intake of antioxidants through diet and supplements protects against oxidant stress and helps maintain the normal function of the vascular system and brain.

Homocysteine is a reliable marker for cardiovascular health and also provide an important clue about the health of your bones. A study from Harvard and Tufts showed that women with the highest levels of homocysteine had almost twice the risk of hip fracture compared to women with the lowest levels. Among men the association was even more pronounced. Specifically, those men with high homocysteine levels had nearly four times the risk of hip fracture as the men whose levels were low. Elevated homocysteine levels appear to be a strong and independent risk factor for osteoporotic fractures in older men and women.

Studies have shown that homocysteine levels may be controlled with ample amounts of folic acid. However, folic acid may sometimes mask a vitamin B₁₂ deficiency. Thus, adding vitamin B₆ may make folate more effective. A benefit of the present invention is that it contains both folate and Vitamin B₁₂.

Previous studies have shown that the content of vitamins B₁₂ and folic acid in white kidney bean extract may decrease during long term use of the extract. The compositions of the present invention, which include folic acid for women and vitamin B₁₂ for men and women, show no decrease of either folic acid or vitamin B₁₂ within these parameters of this study.

One of ordinary skill in the art will understand and appreciate the dosages and timing of the dosages to be administered to a subject in need thereof. The doses and duration of treatment may vary, and may be based on assessment by one of ordinary skill in the art based on monitoring and measuring improvements such as but not limited to weight, BMI, cholesterol levels, blood pressure or combinations thereof. This assessment may be made based on outward physical signs of improvement, or on internal physiological signs or markers. The doses may also depend on the condition or disease being treated, the degree of the condition or disease being treated, the physical characteristic being targeted (for example weight, BMI, cholesterol levels, blood pressure or combinations thereof) and further on the age, weight, body mass index and body surface.

In some embodiments, the compositions described herein may be administered once per day or multiple times per day, such as 1 to 5 doses, twice per day or three times per day.

Specific modes of administration of the compositions described herein will depend on the indication. The selection of the specific route of administration and the dose regimen may be adjusted or titrated by the clinician according to methods known to the clinician in order to obtain the optimal clinical response. The amount of compound to be administered may be that amount which is therapeutically effective. The dosage to be administered may depend on the characteristics of the subject being treated, e.g., the particular animal or human subject treated, age, weight, body mass index, body surface area, health, types of concurrent treatment, if any, and frequency of treatments, and can be easily determined by one of skill in the art (e.g., by the clinician).

The therapeutically effective amounts the compositions described herein may be administered. Each of the compositions described herein may be used in any of the methods or dosage regimens described herein.

In some embodiments, administering a therapeutically effective amount of the compositions described herein may include administering the composition in an immediate release form or a slow or controlled release form as described herein.

Compositions described herein in a solid dosage may include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention.

It is also known in the art that the active ingredients may be contained in such compositions with pharmaceutically or neutraceutically acceptable diluents, fillers, disintegrants, binders, lubricants, surfactants, hydrophobic vehicles, water-soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, compositions may be suitable for oral administration such as, for example, a solid oral dosage form or a capsule, and in certain embodiments, the composition may be a tablet. Such tablets may include any number of additional agents such as, for example, one or more binder, one or more lubricant, one or more diluent, one or more surface active agent, one or more dispersing agent, one or more colorant, and the like. Such tablets may be prepared by any method known in the art, for example, by compression or molding. Compressed tablets may be prepared by compressing in a suitable machine the ingredients of the composition in a free-flowing form such as a powder or granules, and molded tablets may be made by molding in a suitable machine a mixture of the powdered compound moistened with an inert liquid diluent. The tablets, of some embodiments, may be uncoated and, in other embodiments, they may be coated by known techniques.

In other embodiments, the compositions may be provided in a dragee core with suitable coatings. In such embodiments, dragee cores may be prepared using concentrated sugar solutions, which may optionally contain gum arabic, talc, polyvinyl pyrrolidone, carbopol gel, polyethylene glycol, and/or titanium dioxide, lacquer solutions, and suitable organic solvents or solvent mixtures. In some embodiments, dyestuffs or pigments may be added to the tablets or dragee coatings for identification or to characterize different combinations of active compound doses. In yet other embodiments, therapeutically effective amounts of the composition prepared for oral administration may include, but are not limited to, push-fit capsules made of gelatin, as well as soft, sealed capsules made of gelatin and a plasticizer, such as glycerol or sorbitol. The push-fit capsules may contain the active ingredients in admixture with filler such as, e.g., lactose, binders such as, e.g., starches, and/or lubricants such as, e.g., talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All compositions for oral administration should be in dosages suitable for such administration.

In embodiments in which the tablets and dragee cores are coated, the coatings may delay disintegration and absorption in the gastrointestinal tract and thereby providing a sustained action over a longer period. Additionally, such coatings may be adapted for release of the composition in a predetermined pattern (e.g., in order to achieve a controlled release composition) or it may be adapted not to release the active compound until after passage of the stomach (enteric coating). Suitable coatings encompassed by such embodiments may include, but are not limited to, sugar coating, film coating (e.g., hydroxypropyl methylcellulose, methylcellulose, methyl hydroxyethy 1 cellulose, hydroxypropyl cellulose, carboxymethylcellulose, acrylate copolymers, polyethylene glycols and/or polyvinylpyrrolidone), or an enteric coating (e.g., methacrylic acid copolymer, cellulose acetate phthalate, hydroxypropylmethylcellulose phthalate, hydroxypropyl methylcellulose acetate succinate, polyvinyl acetate phthalate, shellac, and/or ethyl cellulose). Furthermore, a time delay material such as, for example, glyceryl monostearate or glyceryl distearate may be incorporated into the coatings of some embodiments. In still other embodiments, solid tablet compositions may include a coating adapted to protect the composition from unwanted chemical changes, for example, to reduce chemical degradation prior to the release of the active substance.

In some embodiments, the compositions may be prepared as suspensions, solutions or emulsions in oily or aqueous vehicles suitable for injection. In such embodiments, such liquid compositions may further include formulatory agents such as suspending, stabilizing and or dispersing agents formulated for parenteral administration. Such injectable compositions may be administered by any route, for example, subcutaneous, intravenous, intramuscular, intra-arterial or bolus injection or continuous infusion, and in embodiments in which injectable compositions are administered by continuous infusion, such infusion may be carried out for a period of about 15 minutes to about hours. In certain embodiments, compositions for injection may be presented in unit dosage form, e.g., in ampoules or in multi-dose containers, with an added preservative.

In other embodiments, the compositions described herein may be formulated as a depot preparation, and such long acting compositions may be administered by implantation (for example subcutaneously or intramuscularly) or by intramuscular injection. Depot injections may be administered at about 1 to about 6 months or longer intervals. In some embodiments, the frequency of doses of the compositions described herein administered by depot injection may be once a month, every three months, or once a year. The compounds may be formulated with suitable polymeric or hydrophobic materials (for example, as an emulsion in an acceptable oil) or ion exchange resins, or as sparingly soluble derivatives, for example, as a sparingly soluble salt.

In still other embodiments, compositions may be formulated for buccal or sublingual administration. In such embodiments, the pharmaceutical compositions may be prepared as chewable tablets, flash melts or lozenges formulated in any conventional manner.

In yet other embodiments, compositions may be formulated for administration by inhalation. In such embodiments, pharmaceutical compositions may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In further embodiments, compositions may be administered intranasally or by inhalation including, but not limited to, an intranasal spray or by pulmonary inhalation with an appropriate carrier. One suitable route of administration is a depot form formulated from a biodegradable suitable polymer, e.g., poly-D,L-lactide-coglycolide as microcapsules, microgranules or cylindrical implants containing dispersed composition.

In further embodiments, compositions may be formulated in rectal compositions such as suppositories or retention enemas, e.g., containing conventional suppository bases such as cocoa butter or other glycerides.

In some embodiments, compositions may be formulated for transdermal administration. For example, such pharmaceutical compositions may be prepared to be applied to a plaster or applied by transdermal, therapeutic systems supplied to the subject. In other embodiments, compositions for transdermal administration may include a suitable solid or gel phase carriers or excipients such as, but are not limited to, calcium carbonate, calcium phosphate, various sugars, starches, cellulose derivatives, gelatin, and polymers such as, e.g., polyethyleneglycols. In some embodiments, compositions may be administered alone as a single agent. In other embodiments, the compositions may be administered in combination with one or more other active ingredients, such as, for example, adjuvants, protease inhibitors, or other compatible agents or compounds where such a combination is seen to be desirable or advantageous in achieving the desired effects of the methods described herein.

The compositions described herein may be prepared, packaged, or sold in bulk as a single unit dose or as multiple unit doses and may be administered in the conventional manner by any route where they are active. For example, the compositions may be administered orally, ophthalmically, intravenously, intramuscularly, intra-arterially, intramedullary, intrathecally, intraventricularly, transdermally, subcutaneously, intraperitoneally, intravesicularly, intranasally, enterally, topically, sublingually, rectally, by inhalation, by depot injections, or by implants or by use of vaginal creams, suppositories, pessaries, vaginal rings, rectal suppositories, intrauterine devices, and transdermal forms such as patches and creams. Specific modes of administration will depend on the indication. The selection of the specific route of administration and the dose regimen may be adjusted or titrated by the clinician according to known methods in order to obtain the optimal clinical response. All of the methods described herein may be carried out by administering the compositions by any such route for administration described herein. Additionally, the compositions may be delivered by using any such route of administration for all of the dosage regimens described herein. The compositions and amounts of non-active ingredients in such a composition may depend on the amount of the active ingredient, and on the size and shape of the tablet or capsule. Such parameters may be readily appreciated and understood by one of skill in the art.

In some embodiments, the compounds may be formulated readily by combining these compounds with pharmaceutically acceptable carriers well known in the art. Such carriers enable the compounds to be formulated as tablets, pills, dragees, capsules, liquids, gels, syrups, slurries, suspensions and the like, for oral ingestion by a subject to be treated. Compositions for oral use may be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethylcellulose, sodium carboxymethylcellulose, and polyvinylpyrrolidone (PVP). In some embodiments, disintegrating agents may be added, such as, but not limited to, the cross-linked polyvinyl pyrrolidone, agar, or alginic acid or a salt thereof such as sodium alginate.

In some embodiments, the pharmaceutical composition may include a diluent in an amount from about 20% to about 50% by weight of said composition; optionally, a second diluent in an amount from about 10% to about 30% by weight of said composition; optionally, a disintegrant in an amount from about 2% to about 6% of said composition; optionally, a lubricant in an amount from about 0.01% to about 2% of said composition. In further embodiments, the pharmaceutical composition may include any amount or combination of microcrystalline cellulose, mannitol, croscarmellose sodium, crospovidone, croscarmellose magnesium stearate, or combination thereof. In some embodiments, the pharmaceutical composition may include microcrystalline cellulose, mannitol, croscarmellose sodium, magnesium stearate, or a combination thereof. In other embodiments, the composition may include microcrystalline cellulose in an amount from about 20% to about 50% by weight of said composition; mannitol in an amount from about 10% to about 30% by weight of said composition; crospovidone in an amount from about 2% to about 6% of said composition; magnesium stearate in an amount from about 0.01% to about 2% of said composition.

In some embodiments, the compositions described herein may further include one or more diluent, one or more disintegrant, one or more lubricant, one or more pigment or colorant, one or more gelatin, one or more plasticizer and the like.

Embodiments of the invention are not limited to any particular agent encompassed by the classes of agents described above, and any agent that falls within any of these categories may be utilized in embodiments of the invention. Non-limiting examples of such agents are provided for clarity. Any of the secondary agents described above may be useful in embodiments of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

In some embodiments, the compositions described herein may further include one or more diluent, one or more disintegrant, one or more lubricant, one or more pigment or colorant, one or more gelatin, one or more plasticizer and the like.

Embodiments of the invention are not limited to any particular agent encompassed by the classes of agents described above, and any agent that falls within any of these categories may be utilized in embodiments of the invention. Non-limiting examples of such agents are provided for clarity. Any of the secondary agents described above may be useful in embodiments of the invention.

In some embodiments, the Ceratonia siliqua extract may be obtained from carob kernels. In some embodiments, the carob kernels are peeled without damaging the endosperm and the germ using special processes (1): (i) Acid peeling process: The kernels are treated with sulfuric acid at a certain temperature to carbonize the seed coat. The carob gum produced with acid peeling process is whitish. (ii) Thermal peeling process: The kernels are roasted in a rotating furnace where the seed coat ‘pops off’ from the rest. The carob gum produced with thermal peeling process is somewhat darker. The isolated endosperm is ground to fine particle size powder, which is the locust (carob) bean gum. The gum may be further clarified as follows: The carob bean gum is dispersed in water and dissolved by heating. This solution is filtered to remove insoluble material. From this clear solution, the carob bean gum is precipitated with isopropanol or ethanol. The precipitate is then filtered, dried and ground to fine particle size powder.

In some embodiments, The specifications set forth at the 67t JECFA (2006) are listed below in Table 1:

TABLE 1

| Parameter | Specification |
|---|---|
| Description | White to yellowish white, nearly odorless powder |

Identification

| | |
|---|---|
| Solubility (6) | Transfer a known amount of the sample into a flask |
| | containing known amount of ethanol, shake for no |
| | less than 30 sec and no more than 5 min - Insoluble |
| | in ethanol |
| Gel formation | An aqueous dispersion forms a gel with small |
| | amounts of sodium borate TS |
| Viscosity | Transfer 2 g of the sample into a 400-mL beaker and |
| | moisten thoroughly with about 4 mL of isopropanol. |
| | Add 200 mL of water with vigorous stirring until |
| | the gum is completely and uniformity dispersed. An |
| | opalescent, slightly viscous solution is formed. |
| | Transfer 100 mL of this solution into another |
| | 400-mL beaker. Heat the mixture in a boiling water |
| | bath for about 10 min and cool to room temperature. |
| | There is an appreciable increase in viscosity. |
| Gum | Galactose and mannose should be present |
| constituents (6) | |
| Microscopic | Disperse a sample of the gum in an aqueous solution |
| examination | containing 0.5% opdine and 1% potassium iodide |
| | on a glass slide and examine under a microscope. |
| | Carob bean gum contains long stretched tubiform |
| | cells, separated or slightly interspaced. Their |
| | brown contents are much less regularly formed |
| | than in Guar gum. |

Purity

| | |
|---|---|
| Loss on Drying (6) | Not more than 14.0% (105°, 5 h) |
| Total ash (6) | Not more than 1.5% |
| Acid-insoluble | Not more than 4.0% |
| matter (6) | |
| Protein (7) | Kjeldahl method: Not more than 7.0% |
| Starch (7) | To a 1 in 10 dispersion of the sample add a few |
| | drops of iodine TS; no blue color is produced |
| Ethanol and | Not more than 1%, singly or in combination |
| Isopropanol (7) | |
| Lead (6) | Not more than 2 mg/kg |
| Microbiological | Total plate count (6): Not more than 5,000 CFU/g |
| criteria (7) | E. coli (7): Negative in 1 g |
| | Salmonella (6): Negative in 25 g |
| | Yeast and moulds (7): Not more than 500 CFU/g |

Safety Evaluation

Absorption, Distribution, Metabolism and Excretion

The galactomannans in locust bean gum are resistant to gastric juices and enzymes in the human GI tract regardless of the specific galactose:mannose ratio. Since it is not digested, little or no absorption of locust bean gum/locust bean gum related species is expected into the systemic circulation. As evidenced in preclinical in vivo studies, almost all of locust bean gum is expected to be excreted unchanged in the feces.

Toxicity Studies

Non-Clinical locust bean gum has shown to have very low acute oral toxicity either when administered in the diet or through oral gavage in several animal species. Oral LD50 for gavage in mouse, hamster, rat and rabbit have been reported to be 13, 10, 5 and 9 g/kg body weight, respectively. In sub-acute toxicity studies in (i) rats—2400 mg/kg bw/day for 36 days and 12000 mg/kg bw/day for 2 weeks, (ii) mice—20000 mg/kg bw/day for 2 weeks, and (iii) neonatal piglets—1000 mg/kg/day for 12 days, no locust bean gum related adverse events were reported. In a sub-chronic study with rats (male and female) that were dosed with locust bean gum at 0, 900, 1800 and 4500 mg/kg bw/day for 90 days, no treatment related effects except elevation in glucose levels to some extent at the highest dose were reported. In a chronic toxicity study with 50 Fisher 344 rats of both sexes exposed to locust bean gum doses of up to 2500 mg/kg bw/day for 103 weeks, no significant treatment-related effects on gross or microscopic pathology were reported. A randomized controlled crossover study in nineteenth healthy volunteers (with and without 50 g of carob fiber) evaluating delayed effects of carob fiber consumption showed an increase in total and acylated plasma ghrelin accompanied by enhanced lipid metabolism the day subsequent to carob fibre ingestion, which indicates higher lipid utilization and suppressed lipolysis. Posptprandial elevation of glucose levels were noted the day after carob fiber consumption.

Genotoxicity Studies

locust bean gum was shown to be not genotoxic in vitro in studies performed using Saccharomyces cerevisiae and Salmonella typhimurium both in the presence and absence of metabolic activation from adult mouse (ICR), rat (Sprague-Dawley) and monkey (Macaca mulatta) (5).

A separate study reported negative results for locust bean gum in in vitro bacterial gene mutation test and in vitro chromosome aberration test.

No evidence of in vitro genotoxicity was found based on various Ames tests using S. cerevisiae (D-3) and S. typhimurium (TA11530 and G46). In addition, the Ames test performed for locust bean gum in the presence and absence of liver S9 fraction preparation from make Sprague-Dawley rats and male Syrian hamsters (S. typhimurium strains TA97, TA98, TA100, TA102, TA104, TA1535, TA1537 and TA1538) all gave negative results.

In vivo mutagenicity tests using locust bean gum including a host-mediated microbial test, a chromosome aberration test and a dominant lethal test, were all negative. It should be noted that, since locust bean gum undergoes negligible hydrolysis, it is not expected to be systemically available to contact target cells to result in genotoxicity.

Carcinogenicity, Reproductive Toxicity and Teratogenicity Studies

All the available studies indicate that locust bean gum is not carcinogenic, teratogenic or that it causes reproductive toxicity.

Other Information Pertinent to Use in Humans

Its important thickening properties have been extensively employed in infant formulas in the context of dietary management of infant regurgitation for more than 20 years.

The Joint FAO/WHO Expert Committee on Food Additives (JEFCA) has authorized the use of locust bean gum as a thickener in infant formula at a level of 0.1 g/100 mL.

locust bean gum is also approved in infant formulae in the EU at the same level by the Scientific Committee on Food (SCF). Additionally, the SCF has approved the use of locust bean gum at levels up to 1 g/100 mL in weaning foods.

In Australia and New Zealand, locust bean gum is approved for use in infant formula at up to 0.1 g/100 mL and up to 1 g/100 mL in infant foods.

In the US, although an infant formula containing locust bean gum is not on the market, the GRAS status (21CFR184.1343) and reviews of the Centre for Food Safety & Applied Nutrition of FDA, locust bean gum can be used in infant formula at levels NMT 5 g/L.

In China, the food and safety standard on additives (GB 2760-2011) allows an locust bean gum level for infant and young children formula up to 0.7 g/100 mL.

Currently, AR formulas with an locust bean gum level of up to 0.5 g/100 mL and intended for infants from birth onwards are on the market world-wide in at least 60 countries.

Thickening infant formulas with locust bean gum (E410) can negatively influence the availability of calcium, iron, and zinc. This effect could be compensated, however, by increasing the amount of essential elements in the infant food sample.

A prospective, randomized, double-blinded, controlled, multi-center, crossover study comparing infant formulas including 115 exclusively formula-fed infants (age 2 weeks to 5 months) was conducted in France. The infants were fed 2 different formulas that contained locust bean gum for 4 weeks with a daily intake of locust bean gum (through the formula) was ˜20 g. No serious adverse events were noted.

A cross-over study assessing the hypolipidemic effect of locust bean gum in 18 familial hypercholesterolemic (FHC) patients and 10 normal subjects who were arbitrarily assigned to 2 groups that were fed identical food products with and without locust bean gum (8-30 g/day), showed no serious side effects over a period of 16 weeks.

Gadolinium-enhanced MRE (magnetic resonance enteroclysis) with locust bean gum (locust bean gum) was found to be very efficient in the detection and follow-up in the intestinal and extraintestinal findings of Crohn's disease.

Despite the broad use of carob gum allergic reactions are seldom reported. Two cases of definite allergic reaction to carob pod is reported in the literature.

No serious reactions to locust bean gum have been noted till date in humans.

History of Use

Locust bean pods were utilized as cattle feed for a very long time. Start of human use of locust bean gum can be dated to the first-century Greeks who used the viscous locust bean gum as a laxative (4). locust bean gum is considered as the first galactomannan used as an additive in several industries including the pharmaceutical and food industries, specifically to increase viscosity, stabilize emulsions and replace fat in many food products. It is typically added in cream cheese to impart richness and spreadability. It is also used in ice cream and yoghurt preparations to improve mouth feel. locust bean gum has been used to form edible films/coatings due to its biocompatibility and biodegradability to enhance the shelf-life of fruits, vegetables and meat products. Given the stability of locust bean gum solutions at a wide range of pH values as well as heat, it is a popular choice for a thickening and stabilizing agent in beverages. locust bean gum has also been used in bakery products to improve final texture and in noodles to improve dough rheology. In the United States, locust bean gum has been used as a stabilizer and thickener in the following foods as shown in Table 2:

TABLE 2

| | Max Use Level |
|---|---|
| Food Category | (%) |
| Baked goods & baking mixes | 0.15 |
| Non-alcoholic beverages & beverage bases | 0.25 |
| Cheeses | 0.80 |
| Gelatins, puddings & fillings | 0.75 |
| James and jellies | 0.75 |
| All other foods | 0.50 |

Regulatory Status

Locust bean gum meets the specifications of the United States Food Chemicals Codex, 3^{rd} Ed. (1981) and is recognized as GRAS by the US FDA (21CFR184.1343 Locust (carob) bean gum). It should be noted that according to the FDAs Inactive Ingredients Database (updated Oct. 24, 2013), locust bean gum has been used in two different commercial products, a chewable bar (locust bean gum at 40 mg) and an extended release oral tablet (locust bean gum at 74.25 mg).

Locust bean gum (E 410) is listed in Annex I of the European Parliament and Council Directive 95/2/EC of 20 Feb. 1995 on food additives, and may be used at “quantum satis” in many food categories.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### EXAMPLES

The following Examples serve to further illustrate the present invention and are not to be construed as limiting its scope in any way.

### Example 1

### Weight Loss

Six obese individuals (3 women and 3 men) with a BMI greater than 30 kg/m2 and with untreated hypertension and high blood lipids, volunteered to participate in a study to examine the efficacy of a green tea extract and phaseolamin-containing supplement in weight reduction. The supplement comprises 200 mg, white kidney bean extract 50 mg, locust bean gum extract, and 100 mg green tea extract, given twice daily, as a capsule half an hour before lunch and dinner. This composition is referred to as the Test Composition throughout the following examples. The individuals were obese and had high plasma-lipid levels, but were otherwise healthy individuals between 43 to 65 years of age, with no known disease and taking no medication. Patients on a weight loss regimen, restricted diet, smokers, patients included in other studies and patients taking diet supplements or vitamins were excluded. Lipids and nutritional blood parameters were measured at baseline and at the end of the study. Excretion of fat in feces was measured at the end of study and after a wash out period of one week. The study lasted for 8 weeks and the results appear in Table 3.

TABLE 3
WEIGHT LOSS FOLLOWING 8-WEEK ADMINISTRATION
OF TEST COMPOSITION

| | Weight in kilogram | Weight in kilograms | Weight in |
|---|---|---|---|
| Patients | at start | after 8 weeks | kilogram lost |
| Patient 1 (PI) | 104.7 | 98.1 | 6.6 |
| Patient 2 (P2) | 108.3 | 101.4 | 6.9 |
| Patient 3 (P3) | 98.4 | 90.2 | 8.2 |
| Patient 4 (P4) | 124.2 | 118.1 | 6.1 |
| Patient 5 (P5) | 143.7 | 136.7 | 7.0 |
| Patient 6 (P6) | 181.4 | 174.3 | 7.1 |

Patients 1, 2 and 3 are female; Patients 4, 5 and 6 are male. The subjects were asked to maintain their own diet and not change their food intake or exercise level. Food intake did change most likely due to the effect of the extract of the green tea. According to weekly diet analyses of food diaries, the following changes took place in each individual. See Table 4.

TABLE 4
CALORIC INTAKE FOLLOWING 8-WEEK ADMINISTRATION
OF TEST COMPOSITION

| Pa- | Week | Week | Week | Week | Week | Week | Week | Week |
|---|---|---|---|---|---|---|---|---|
| tient | 1 | 2 | 3 | 4 | 5 | 6 | 7 | 8 |
| P1 | 2300 | 2240 | 2100 | 1980 | 1760 | 1720 | 1730 | 1720 |
| P2 | 2890 | 2740 | 2710 | 2680 | 2450 | 2340 | 2280 | 2260 |
| P3 | 3200 | 3200 | 3100 | 2940 | 2780 | 2430 | 2400 | 2380 |
| P4 | 2430 | 2200 | 1980 | 1870 | 1780 | 1640 | 1720 | 1680 |
| P5 | 1420 | 1400 | 1460 | 1620 | 1520 | 1320 | 1300 | 1320 |
| P6 | 1900 | 1870 | 1920 | 1860 | 1700 | 1430 | 1540 | 1400 |

The observed changes in caloric intake cannot alone account for the extra weight loss. In a Visual Analogue Scale Hunger Rating Questionnaire, given to the six individuals one week before starting the study, the individuals confirmed they were always hungry at meals. The individuals confirmed that they felt less hungry at meals after administration of the Test Composition. The participants also confirmed that they stopped eating once satisfied and ingested fewer calories when taking the Test Composition. Compared to another study with a white kidney bean extract mixed with locust bean gum extract, participants (P1-P6) felt more satiated faster and therefore ate less. The participants (P1-P6) also lost more weight over a shorter amount of time. In the referenced study with a white kidney bean extract mixed with locust bean gum, the participants lost an average 3.2 kg after 8 weeks compared to an average 5.8 kg for participants P1-P6. (See, Table 3) The weight loss observed for P1-P2 and depicted in Table 1 exceeds the weight loss observed in individuals consuming a composition containing white kidney bean extract mixed with locust bean gum extract.

The weight loss in this study has been induced by influencing the digestive enzyme activity in an attempt to increase the absorption of caloric nutritional components from ingested food items. Well-known examples of such actions include the inhibition of intestinal carbohydrates or lipases due to the mechanisms of the phaseolus vulgaris. Other functional ingredients influence the absorption of nutritional molecules without decreasing intestinal enzyme activity, which may be due to the mechanisms of the Locust Bean Gum extracts. A further effective method of reducing weight in overweight and obese individuals can be accomplished by reduction in food intake, for example, by reducing the desire for food through appetite reducing functional ingredients. In this study the appetite reducing effects are most likely caused by the ingredient of the green tea extract and the composition of these three ingredients. Locust Bean Gum extract is suppressing the hunger hormone ghrelin making individuals less hungry at meals.

### Example 2

### Improved Lipid Profile

The effects of various supplements on plasma lipid profile were studied in a group of individuals using the composition of a supplement containing a white kidney bean extract mixed with locust bean gum. The effects of the Test Composition, composition with a white kidney bean extract mixed with locust bean gum, and placebo (containing no active ingredients (i.e., no white kidney bean extract, locust bean gum extract or green tea extract) on lipid profile are presented in Table 5.

TABLE 5
Effect on Plasma Lipid Profile

| Test | Total | Triglycerides | LDL | HDL | LDL/HDL |
|---|---|---|---|---|---|
| product | cholestrol | (mmol/l) | (mmol/l) | (mmol/l) | ratio |
| start | 6.9 | 1.9 | 4.7 | 1.1 | 3.7 |
| 2 months | 6.1 | 1.8 | 4.3 | 1.2 | 3.8 |

### Example 3

### Body Composition

In the six individuals receiving the Test Composition, each participant's body composition and waist circumference was measured. Body compositions was determined using infrared spectroscopy (NIR, Futrex 5000, Gaithersburg). Waistline measurements are performed at the upper spina iliaca anterior superior underneath the umbilicus. The Test Composition was well tolerated and the participants reported no side effects. The results are shown in Table 6.

TABLE 6
INDICIA OF WEIGHT LOSS IN SUBJECTS
RECEIVING THE TEST COMPOSITION.

| | BMI | Body Fat | Waist |
|---|---|---|---|
| | (kg/M²) | (%) | (cm) |
| Start | 36.0 | 37.3 | 109.2 |
| 8 weeks | 32.1 | 31.4 | 103.4 |

The NIR method is based on the principle that the degree of near infrared scattering is related to the composition of the substance through which the near infrared light passes. As such, the NIR method is considered a direct measure of body fat. The Futrex 5000 apparatus consists of a monochromatic wave emitter and a fiber optic probe, which both conducts radiation from the emitter to a site selected on the body (biceps) and detects interactive radiation. The difference between the amount of light absorbed at two wavelengths (940 and 950 nm) is used to calculate the percentage body fat in the tested (representative) locations. The wavelengths must be chosen in a region of the spectrum sensitive to differences in fat levels. Measurements at the midpoint of the biceps show good correlation to underwater weighing. No correction for physical activity was made in this study.

### Example 4

### Blood Pressure

The effect of the Test Composition on the blood pressure of 3 hypertensive men and 3 hypertensive women were measured in an 8 week study. The results are presented in Table 7. The diastolic and systolic blood pressure decreased to a greater degree after 8-week administration of the Test Composition, See Table 7.

TABLE 7
EFFECTS OF SYSTOLIC AND DIASTOLIC BLOOD PRESSURE
IN SUBJECTS USING THE TEST COMPOSITION

| Test | Systolic BP | Diastolic BP |
|---|---|---|
| Composition | (mmHg) | (mmHg) |
| Start | 148.6 | 95.5 |
| 8 weeks | 137.0 | 85.6 |

What is claimed:

1. A composition comprising a white kidney bean extract; a locust bean gum extract and a green tea extract.

2. The composition of claim 1, wherein the white Kidney bean extract is Phaseolus vulgaris.

3. The composition of claim 1, wherein the Ceratonia siliqua extract is Locust bean gum.

4. The composition of claim 1, wherein the green tea extract is Camellia sinensis.

5. The composition of claim 1, wherein the green tea extract is decaffeinated.

6. The composition of claim 1, wherein the white kidney bean extract, locust bean gum extract, the green tea extract, or any combination thereof is water soluble.

7. The composition of claim 1, further comprising a pharmaceutically acceptable carrier.

8. The composition of claim 7, wherein the pharmaceutically acceptable carrier is calcium sulfate.

9. The composition of claim 1, wherein the white kidney bean extract; locust bean gum extract; and green tea extract is encapsulated.

10. The composition of claim 9, wherein the white kidney bean extract; locust bean gum extract; and green tea extract is encapsulated in vegetable capsule.

11. The composition of claim 10, wherein the vegetable capsule has a disintegration time that is less than or equal to about 45 minutes when administered orally.

12. The composition of claim 1, further comprising a blueberry extract, mint extract, ginger extract, vitamin B₁₂, Vitamin B₆, folic acid or any combination thereof.

13. The composition of claim 1, wherein the composition is formulated in an immediate release form.

14. The composition of claim 1, wherein the composition is formulation in a slow release form.

15. The composition of claim 1, wherein the white kidney bean extract is enriched for phaseolamin.

16. The composition of claim 1, wherein the locust bean gum is seed coated.

17. The composition of claim 1, wherein the green tea is enriched for catechol.

18. The composition of claim 17, wherein the green tea extract comprises about 20% to about 30% catechol by weight.

19. The composition of claim 17, wherein the green tea extract contains about 25% catechol by weight.

20. The composition of claim 1, wherein the green tea extract is enriched for polyphenols.

21. The composition of claim 20, wherein the green tea extract contains about 10% to about 20% polyphenols by weight.

22. The composition of claim 20, wherein the green tea extract contains about 10% to about 17% polyphenols by weight.

23. The composition of claim 20, wherein the green tea extract contains about 17% polyphenols by weight.

24. The composition of claim 1, wherein the green tea extract contains about 5% to about 10% caffeine by weight.

25. The composition of claim 1, comprising about 100 milligrams and about 1,000 milligrams the white kidney bean extract

26. The composition of claim 1, further comprising about 100 milligrams to about 1,000 milligrams phaseolamin.

27. The composition of claim 26, comprising about 200 milligrams of phaseolamin

28. The composition of claim 1, comprising about 200 mg of white kidney bean extract.

29. The composition of claim 1, comprising about 25 milligrams to about and 250 milligrams of locust bean gum extract.

30. The composition of claim 1, comprising about 50 milligrams of locust bean gum extract.

31. The composition of claim 1, comprising about 10 milligrams to about 500 milligrams of green tea extract.

32. The composition of claim 1, comprising about 100 milligrams of green tea extract.

33. A method for promoting weight loss comprising the step of administering a therapeutically effective amount of the composition of claim 1 to a subject in need thereof.

34. The method of claim 33, wherein the composition is administered orally.

35. The method of claim 33, wherein said subject has a BMI between about 25 kg/m² and 30 kg/m².

36. The method of claim 33, wherein said subject has a BMI greater than 30 kg/m².

37. A method for reducing elevated blood pressure comprising the step of administrating a therapeutically effective amount of the composition of claim 1 to a subject in need thereof.

38. The method of claim 37, wherein the composition is administered orally.

39. The method of claim 37, wherein the administration of said composition reduces systolic and diastolic blood pressure by at least about 5%.

40. A method for altering a plasma lipid profile comprising the step administrating a therapeutically effective amount of the composition of claim 1 to a subject in need thereof.

41. The method of claim 40, wherein administration of said composition reduces plasma low-density lipoprotein (LDL), increases plasma high-density lipoprotein (HDL), increases the ratio HDL to LDL, reduces total plasma cholesterol or any combination thereof.

42. The method of claim 41, wherein said subject has elevated plasma low-density lipoprotein (LDL), depressed plasma high-density lipoprotein HDL, a depressed HDL to LDL ratio or any combination thereof.

43. A method of promoting gut health, comprising the step of administering a therapeutically effective amount of the composition of claim 1 to a subject in need thereof.

44. The method of claim 43, wherein the composition is administered orally.

45. The method of claim 43, wherein the subject is diagnosed with irritable bowel syndrome.

46. A method of preventing, treating, and/or ameliorating the symptoms of irritable bowel syndrome, comprising the step of administering a therapeutically effective amount of the composition of claim 1 to a subject in need thereof.

47. The method of claim 46, wherein the composition is administered orally.

48. A method of making the composition of claim 1, comprising combining the white kidney bean extract, locust bean gum extract, and green tea extract, wherein the white kidney bean extract, locust bean gum extract, and green tea extract, or any combination thereof, is water soluble.
